(19) United States
(12) Patent Application Publication
(10) Pub. No.: US 2021/0161915 A1
(43) Pub. Date: Jun. 3, 2021

(54) GABA-A ANTAGONISTS FOR TREATING SUBSTANCE WITHDRAWAL DISORDERS

(71) Applicant: ASARINA PHARMA APS, Köpenhamn (DK)

(72) Inventor: Torbjörn Bäckström, Umeå (SE)

(73) Assignee: ASARINA PHARMA APS, Köpenhamn (DK)

(21) Appl. No.: 17/045,241

(22) PCT Filed: Apr. 5, 2019
(86) PCT No.: PCT/EP2019/058607

§ 371 Date: Oct. 5, 2020

(30) Foreign Application Priority Data
Apr. 5, 2018 (SE) 1850385-4
Jan. 22, 2019 (SE) 1950069-3

(51) Int. Cl. A61K 31/573; A61K 31/568; A61K 31/167; A61K 31/192; A61K 31/616; A61K 31/196; A61K 31/4045; A61K 31/404; A61K 31/454; A61K 31/4196; A61K 31/422; A61P 25/06; A61P 25/30

(52) U.S. Cl. CPC A61K 31/573; A61K 31/568; A61K 31/167; A61K 31/192; A61K 31/616; A61K 31/196; A61P 25/30; A61K 31/404; A61K 31/454; A61K 31/4196; A61K 31/422; A61P 25/06; A61K 31/4045

(57) ABSTRACT

The present disclosure provides GABA_{A} receptor modulating steroid antagonists for use in treatment of substance withdrawal disorders as well as methods for treating said disorders and pharmaceutical compositions for use in treatment of said disorders.

### TECHNICAL FIELD

The present disclosure provides GABA_{A} receptor modulating steroid antagonists for use in treatment and/or prevention of a substance withdrawal disorder, use of GABA_{A} receptor modulating steroid antagonists for the manufacture of a medicament for the treatment and/or prevention of a substance withdrawal disorder, methods for treating said disorders as well as pharmaceutical compositions for use in treatment of said disorders.

### BACKGROUND

Substance withdrawal disorders may be common, paroxysmal, and/or chronic. In many ways they are clearly different diseases, yet there are some pathophysiological overlaps, and overlaps in clinical symptomatology, particularly with regard to visual and other sensory disturbances, pain, depression, headache, alterations of consciousness, seizure(s), and dizziness.

Continuous brain exposure to positive GABA_{A} receptor modulators such as barbiturates, benzodiazepines and alcohol, induce tolerance development (Wolf & Griffiths 1991; Barnes 1996). Long brain elevated exposure to endogenous compounds like positive GABA_{A} receptor modulating steroids (such as 3alpha-hydroxy-5alpha/beta-steroids) has similar effects as benzodiazepines and barbiturates, and may also cause tolerance and thus malfunctioning of the GABA_{A} receptor system (Turkmen et al. 2011). The following themes have hitherto been the main subject of research: downregulation and decreased GABA function after long-term secretion of high amounts of 3alpha-hydroxy-5alpha/beta steroids; reduced benzodiazepine and 3alpha-hydroxy-5beta steroid sensitivity in females; and dependence induction both in males and females (Turkmen et al. 2011).

A continuous elevated exposure to GABA_{A} receptor modulating steroids, such as 3alpha-hydroxy-5alpha/beta-steroids results in a withdrawal effect when the exposure has ended. This phenomenon occurs for example naturally, during menstruation when the production of 3alpha-hydroxy-5alpha/beta-steroids by the corpus luteum of the ovary is interrupted (Laidlaw 1956, MacGregor 2009). This withdrawal phenomenon also occurs after giving birth (post partum) when the 3alpha-hydroxy-5alpha/beta-steroid production by the placenta is interrupted (O'Keane et al. 2011). The same phenomenon has been observed when a period of stress has ended, e.g. when work-related stress is interrupted during weekends. As a response to stress, the adrenals have produced 3alpha-hydroxy-5alpha/beta-steroids (Droogleever Fortuyn et al. 2004). When this production is interrupted, withdrawal symptoms might occur. In relation to interruption of long benzodiazepine use, it is believed that there is an increased risk for seizure(s). Similar increased risk is noted in women with epilepsy at sudden interruption of endogenous production of 3alpha-hydroxy-5alpha/beta-steroids (Bäckström 1976). A similar relation exists between headache/migraine and sudden withdrawal of benzodiazepines or endogenous 3alpha-hydroxy-5alpha/beta-steroids. Specific diagnostic criteria and classification are found in International headache society's “International Classification of Headache Disorders”. IHS ICHD-3 group 8.3 Headache associated with and/or attributed to substance withdrawal and 8.3.3 Headache associated with and/or attributed to steroid withdrawal (IHS 2018).

It is generally recognized that the development of migraine headache depends on the activation of the trigeminovascular system (TGVS). Stimulation of the trigeminovascular system is responsible for the pain process during a migraine episode. Trigeminovascular pain can be modulated through GABAergic mechanisms (Storer et al 2001). Further, midazolam, a benzodiazepine agonist that facilitates GABA_{A} receptor modulation, can dose-dependently inhibit trigeminal neurons activated by pain-producing trigeminovascular input neurons (Andreou et al 2010). Furthermore, the benzodiazepine antagonist flumazenil dose-dependently inhibits the pain reducing effect of midazolam (Storer et al 2004). However, a problem with positive GABA_{A} receptor modulating compounds is that they induce tolerance development and the treatment effect will thus fade off quite rapidly. Worse is that if the treatment is rapidly stopped, a withdrawal effect will appear with increased excitability and withdrawal symptoms. GABA_{A} receptor modulating steroids like allopregnanolone, a neurosteroid and progesterone metabolite is also a positive modulator of the GABA_{A} receptor via an allosteric binding site (Cutrer et al 1995; Drower and Hammond 1988). Allopregnanolone can also induce tolerance similarly as other positive GABA_{A} receptor modulators, with a withdrawal effect when withdrawn (Birzniece et al 2006). During the second part or the menstrual cycle the concentration of allopregnanolone is elevated both in serum and in the brain. Similarly, levels of allopregnanolone and other GABA_{A} modulating steroids are increased during periods of stress. Tolerance will develop and when the steroid is withdrawn a rebound of headache, migraine and seizure(s) may occur (Reddy et al 2012). From studies of women with epilepsy, it has been described that the concentration of allopregnanolone is high enough to decrease the seizure frequency during the early luteal phase but the seizure frequency increases at the withdrawal period during the menstruation (Bäckström 1976).

Continuous elevated exposure to GABA_{A} receptor modulating steroids causes tolerance development and malfunction of the GABA_{A} receptor system in many individuals (Turkmen et al 2011). The GABA_{A} receptor is very sensitive to tolerance development against any kind of positive modulator of the receptor. Tolerance is a known phenomenon against e.g. benzodiazepines and barbiturates (Barnes 1996). The 3alpha-hydroxy-5alpha-steroid allopregnanolone can induce tolerance already after 60-90 minutes of continues exposure (Zhu et al 2004; Birzniece et al 2006).

Allopregnanolone is a potent anesthetic drug, acting via the GABA_{A} receptor by enhancing the effect of GABA on chloride flux. As other anesthetic drugs, acting via the GABA_{A} receptor, such as barbiturates and benzodiazepines, allopregnanolone induce tolerance when exposure is present during longer periods (Zhu et al 2004, Turkmen et al 2011). One way of hindering tolerance development is to antagonize the effect of the GABA_{A} receptor modulating steroid on the GABA_{A} receptor and thus hinder the CNS action of e.g. allopregnanolone and induction of withdrawal (Brust 2004). One way of investigating if a proposed compound has such an antagonizing effect is to use an anesthesia model as the in vivo effect easily can be discovered.

Loss of righting (LoR) reflex and silent second change on EEG are two such models (Vanini et al 2008; Zhu et al 2004). When the positive GABA_{A} receptor modulating effect is inhibited, tolerance development is inhibited and as a resultant of that the withdrawal effect is inhibited.

Upregulation of the alpha4,beta,delta subunit of the GABA_{A} receptor is especially interesting as an upregulation in the brain occurs at the tolerance development to 3alpha-hydroxy-5alpha-pregnan-20-one (Birzniece et al 2006). This specific receptor subtype alpha4,beta,delta is hypersensitive to 3alpha-hydroxy-5alpha/beta-pregnan/Δ4-pregnen-20-one/ol steroids and is believed to be involved in withdrawal phenomena (Smith et al 1998).

The severity of behavioral symptoms in withdrawal disorders is exacerbated by chronic psychosocial stress depending on the duration of the chronic stress. A chronic stress duration of one week is enough to cause a withdrawal effect when the person relaxes e.g. during weekends (Akerman et al 2017). When the continuous stimulation of the GABA_{A} receptor is interrupted, a period of withdrawal occurs. There are several periods in life when a continuous high production of 3alpha-hydroxy-5alpha/beta-steroids occurs. During each menstrual cycle a withdrawal of allopregnanolone occurs prior to the menstruation, when allopregnanolone is no longer produced from the corpus luteum. The first few days of menstruation are thus more sensitive to over activity in excitatory CNS functions. In certain women with migraine, the migraine attacks increase in frequency, are longer and more severe during the menstruation and they seem more refractory to conventional migraine treatment (Vetvik et al 2015; MacGregor 2009). Besides migraine attacks, also the frequency and intensity of seizure(s) in women with epilepsy increase during the menstrual period (Laidlaw 1956; Bäckström 1976). The withdrawal phenomenon of allopregnanolone has also been shown to occur in animal models (Reddy et al 2009; Reddy et al 2012). A prerequisite for a withdrawal phenomenon is that a preceding tolerance has developed. If no GABA_{A} receptor stimulation occurs, tolerance will not develop, and no withdrawal will occur.

Positive GABA_{A} receptor modulating steroids are metabolites of the sex and stress hormones pregnanolone, progesterone, deoxycorticosterone, cortisone and cortisol, known as pregnanolones; as well as the metabolites of testosterone, androstandione and dehydroepiandrosterone, known as androstanes. Positive GABA_{A} receptor modulating steroids have all been the subject of various studies, at least partially elucidating their role in the neurological signal system in mammals. In excess, they are harmful steroids inducing CNS symptoms and disorders. Harmful steroids all have a structural similarity in comprising a 3alpha-hydroxy group, a delta-4-pregnene or a 5alpha or 5beta pregnane steroid body, and a ketone or hydroxy group on position 17, 20 or 21.

3alpha-hydroxy-5alpha/beta-pregnan/Δ4-pregnen-20-one/ol steroids or 3alpha-hydroxy-5alpha/beta-androstan/Δ4-androsten-17-one/ol steroids, such as allopregnanolone, tetrahydrodeoxycorticosterone and androstanediol, have been shown to be important specific enhancers of the GABA_{A} receptor and thus positive GABA_{A} receptor modulating steroids. They bind to the GABA_{A} receptor and act by enhancing the effect of GABA in terms of prolonging duration of the GABA_{A} receptor opening. The GABA_{A} receptors are of several subtypes located in different areas of the brain and related to different CNS disorders and symptoms. Some GABA_{A} receptors are localized within a synapse (intra-synaptic) while others are located outside a synapse (extra-synaptic). GABA_{A} receptor modulating steroids can in physiological concentrations open the extra-synaptic GABA_{A} receptor by themselves (tonic inhibition) but not the intra-synaptic receptors (phasic inhibition). These two types of effects are dependent on different mechanisms on the GABA_{A} receptor and the effects depend in addition on the subunit composition of the receptor. The receptor subtype alpha4,beta,delta is an extra-synaptic subtype with both tonic and phasic effects of 3alpha-hydroxy-5alpha/beta-pregnan-20-one/ol and 3alpha-hydroxy-5alpha/beta-androstan-17-one/ol. No specific GABA_{A} modulating steroid antagonist acting on both binding sites or only inhibiting one of the mechanisms are known. The effect of 3alpha-hydroxy-5alpha/beta-pregnan-20-one/ol or 3alpha-hydroxy-5alpha/beta-androstan-17-one/ol is similar to the effects of both benzodiazepines and barbiturates, i.e. they are all positive modulators of the GABA_{A}receptor. Said steroid compounds, however, have a binding site on the GABA_{A} receptor separate from that of GABA, barbiturates and benzodiazepines.

Disorders caused by the action of endogenously produced 3alpha-hydroxy-5alpha steroids or 3alpha-hydroxy-5beta steroids on the GABA_{A} receptor are characterized and understood. 3alpha-hydroxy-5alpha/beta-steroids can induce tolerance to themselves and to other similar substances after long exposure and withdrawal symptoms occur at withdrawal of the 3alpha-hydroxy-5alpha/beta-steroids.

Prince and Simmons (1993) used a model relying on membrane fractions of whole male rat brain. In this sub-fraction of whole brain homogenate, the authors used the binding of a benzodiazepine, ³H-flunitrazepam, as a model for steroid effect and change of GABA_{A} receptor conformation. This test has been suggested as an indicator of allosteric modulation of the GABA_{A} receptor. This is, however, a general analytical method that does not take into account the specificity depending on the subunit composition or intra or extra synaptic effects or different GABA_{A} modulating steroids. The relationship between the change in flunitrazepam (FNZ) binding and the change in chloride flow during GABA stimulation is, however, uncertain and a change in FNZ-binding cannot be taken as a proof of a change in the chloride flow through the GABA receptor, nor as a proof of a change in GABA_{A} receptor function. The existence of a relationship between change in FNZ-binding and neuronal excitability, is even less clear and such conclusions cannot be drawn from results on FNZ-binding alone. A change in FNZ-binding properties or absence of such change in binding properties does not imply a change or the absence of a change in neural activity or GABA_{A} mediated chloride flow. The GABA_{A} receptor contains several sub-units that can be combined in multiple ways. Interestingly, certain combinations show different recognition sites for steroids and other ligands (Wisden et al. 1992, Olsen 2018).

U.S. Pat. Nos. 5,232,917 and 5,939,545 disclose a number of 3alpha-hydroxy steroids. These disclosures concern the agonistic modulation of the GABA_{A} receptor. In other words, the disclosures are focused on the benzodiazepine-like effect of the 3alpha-hydroxy-5alpha/beta-steroids. All steroids that are positive modulators of the GABA_{A} receptor have the common feature of a 3alpha-hydroxy structure. Steroids with a 3beta-hydroxy structure have never been shown to possess a GABA_{A} receptor positive modulating effect. In all cases where an effective GABA_{A} receptor-modulating effect is noticed, the steroid has a 3alpha-hydroxy group. Further the disclosure does not separate GABA_{A} receptor subunit effects and especially the effect on alpha4,beta,delta subunit combination has not been tested.

WO 99/45931 discloses a GABA_{A} modulating steroid antagonist, namely 3beta-hydroxy-5alpha-pregnan-20-one but does not mention the effect on withdrawal disorders or in different receptor subtypes e.g. the alpha4,beta,delta subtype and does not describe 3beta-hydroxy-5alpha-pregnan-20-one effects on phasic or tonic 3alpha-hydroxy-5alpha/beta-steroid activity in intra- or extra-synaptic receptors. It does not disclose that 3beta-hydroxy-5alpha-pregnan-20-one can inhibit negative symptom provoking effects at withdrawal of a 3alpha-hydroxy-5alpha/beta-androstane-steroid, such as 3alpha-hydroxy-5alpha-androstan-17-ol. Further, there is no disclosure that substance withdrawal disorders at relaxation after a longer period of chronic stress could be treated with 3beta-hydroxy-5alpha-pregnan-20-one.

The GABA_{A} modulating steroid antagonistic effect of 3beta-hydroxy-5alpha-pregnan-20-one against 3alpha-hydroxy-5alpha/beta-pregnan-20-one was first disclosed by Wang et al (2000). In that disclosure, a dose dependent, antagonistic effect of 3beta-hydroxy-5alpha-pregnan-20-one on two of the 3alpha-hydroxy-5alpha/beta-steroids was shown. This document does not mention the possibility to use 3beta-hydroxy-5alpha-pregnan-20-one against disorders caused by androgenic GABA_{A} receptor modulating steroids and the possibility to use 3beta-hydroxy-5alpha-pregnan-20-one as inhibitor at the alpha4,beta,delta activated GABA_{A} receptor and does not disclose 3beta-hydroxy-5alpha-pregnan-20-one effects on phasic or tonic 3alpha-hydroxy-5alpha/beta-steroid activity in intra- or extra-synaptic receptors.

WO 03/059357 discloses the use of certain pregnane steroids in the treatment of CNS disorders. This document does not mention the possibility to use 3beta-hydroxy-5alpha-pregnan-20-one against disorders caused by androgenic GABA_{A} receptor modulating steroids and the possibility to use 3beta-hydroxy-5alpha-pregnan-20-one as inhibitor at the alpha4,beta,delta activated GABA_{A} receptor and does not disclose 3beta-hydroxy-5alpha-pregnan-20-one effects on phasic or tonic 3alpha-hydroxy-5alpha/beta-steroid activity in intra- or extra-synaptic receptors.

3beta steroids can also have effects on the GABA's own effect as disclosed by Wang et al (2002). In this disclosure, the authors show that when maximal stimulation of GABA_{A} receptors is made with GABA, certain 3beta-hydroxypregnane steroids are able to inhibit GABA's own effect. But that certain 3beta-hydroxy steroids would mainly inhibit GABA_{A} receptor modulating steroid effects on GABA gated chloride flux and other steroids would also inhibit GABA's own effect was not discovered or realized. This document does not mention the possibility to use 3beta-hydroxy-5alpha-pregnan-20-one against disorders caused by androgenic GABA_{A} receptor modulation steroids and the possibility to use 3beta-hydroxy-5alpha-pregnan-20-one as inhibitor at the alpha4,beta,delta activated GABA_{A} receptor and does not provide 3beta-hydroxy-5alpha-pregnan-20-one effects on phasic or tonic 3alpha-hydroxy-5alpha/beta-steroid activity in intra- or extra-synaptic receptors.

WO2008/063128 discloses the use of certain pregnane steroids in the treatment of CNS disorders. The disclosure does not mention the possibility to use 3beta-hydroxy-5alpha-pregnan-20-one against disorders caused by androgenic GABA_{A} receptor modulating steroids and the possibility to use 3beta-hydroxy-5alpha-pregnan-20-one as inhibitor at the alpha4,beta,delta activated GABA_{A} receptor and does not provide 3beta-hydroxy-5alpha-pregnan-20-one effects on phasic or tonic 3alpha-hydroxy-5alpha/beta-steroid activity in intra- or extra-synaptic receptors.

It remains a challenge to find specific antagonists of the 3alpha-hydroxy-androstan/pregnane action on the GABA_{A} receptor that have an activity on alpha4,beta,delta subtype combined with low inhibitory effects on GABA itself. In addition, it remains a challenge to find compounds that are physiologically safe and suitable for pharmaceutical use, and which additionally are applicable in physiologically acceptable doses with reasonable time intervals, for treatment of substance withdrawal disorders.

Further objectives, the associated solutions and their advantages follow with the description, examples and claims.

### DESCRIPTION

The term “substance withdrawal disorder” refers to a group of symptoms that occur upon the discontinuation, such as abrupt discontinuation, or decrease of one or more of the following: an endogenously produced substance, exposure to and/or intake of substances, such as medications, recreational drugs and endogenous substances. In particular as used herein, the term “substance withdrawal disorder” refers to a disorder associated with and/or attributed to and/or caused by withdrawal of positive GABA_{A} receptor modulating steroids (GABA_{A} receptor modulating steroid agonists). For the avoidance of any doubt, the clinical symptomatology of the substance withdrawal disorder may vary between individual patients while the underlying disorder is the same. Thus, said disorder may have different clinical manifestation(s). In particular said substance withdrawal disorder may thus manifest as headache, migraine, seizure(s), depression, anxiety and/or panic which is associated with and/or attributed to substance withdrawal.

In order for the symptom(s) of substance withdrawal to occur, the patient has first to develop a substance tolerance or dependence, such as a physical and/or physiological tolerance or dependence. Substance tolerance or dependence may arise upon exposure to a substance for a period of time, for example of a prolonged use of a medicine or exposure to an endogenous substance for a prolonged period of time. Upon discontinuation or decrease of exposure to the substance, such as discontinuation or decrease of substance administration or concentration thereof or discontinuation or decrease of exposure to an endogenous substance, the symptoms of substance withdrawal disorder will manifest.

As explained above, the prerequisite for a withdrawal phenomenon and hence for the development of a substance withdrawal disorder, is that the patient has developed a preceding tolerance to the substance at hand.

As used herein, the term “patient” or “subject” refers to an individual who is exhibits or is at risk of exhibiting symptom(s) of a substance withdrawal disorder.

Substance withdrawal disorders may be common, paroxysmal, and/or chronic. In particular there are a number of endogenous positive GABA-A modulating compounds that are associated with and/or cause tolerance or dependence and therefore symptoms associated with withdrawal when their production stops. In many ways the substance withdrawal disorders may be seen as different diseases, yet there are some significant pathophysiological overlaps, and significant overlaps in clinical symptomatology, particularly with regard to visual and other sensory disturbances, pain, headache, migraine, alterations of consciousness, seizure(s), panic, anxiety, depression and/or dizziness. The disorders are believed to originate from electrical disturbances in the brain. In the present disclosure, there is provided substance withdrawal disorders associated with and/or caused by withdrawal of positive GABA_{A} receptor modulating steroids (also referred to as GABA_{A} receptor modulating steroid agonists or positive allosteric modulators (PAM)). In the present disclosure, it is surprisingly provided that the GABA_{A} receptor stimulation by GABA_{A} receptor modulating steroid agonists can be prevented by using GABA_{A} receptor modulating steroid antagonists (GAMSA). Therefore, such antagonists have been found to be useful in treatment of substance withdrawal disorders.

The substance withdrawal disorder described herein is associated with and/or caused by withdrawal of positive GABA_{A} receptor modulating steroids (GABA_{A} receptor modulating steroid agonists). For the avoidance of any doubt, the clinical symptomatology of the substance withdrawal disorder may vary between individual patients while the underlying disorder is the same. Thus, said disorder may have different clinical manifestations. In particular said, substance withdrawal disorder may thus manifest as headache, migraine, seizure(s), depression, anxiety and/or panic which is associated with and/or attributed to substance withdrawal.

As explained above, the prerequisite for a withdrawal phenomenon and hence for the development of a substance withdrawal disorder, is that the patient has developed a preceding tolerance to the substance at hand.

In a first aspect, the present disclosure provides a GABA_{A} receptor modulating steroid antagonist for use in treatment and/or prevention of a substance withdrawal disorder.

In one embodiment, said substance withdrawal disorders may be associated with, attributed to and/or caused by withdrawal of a positive GABA_{A} receptor modulating steroid (GABA_{A} receptor modulating steroid agonist). Non-limiting examples of positive GABA_{A} receptor modulating steroids include allopregnanolone, androstanediol and, deoxycorticosterone. Said deoxycorticosterone may be tetra-hydro-deoxycorticosterone (THDOC).

In one embodiment said positive GABA_{A} receptor modulating steroid is selected from the group consisting of allopregnanolone, androstanediol, deoxycorticosterone and any combination thereof. The deoxycorticosterone may be tetra-hydro-deoxycorticosterone (THDOC). In one embodiment said positive GABA_{A} receptor modulating steroid is selected from the group consisting of allopregnanolone, deoxycorticosterone and any combination thereof. The deoxycorticosterone may be tetra-hydro-deoxycorticosterone (THDOC).

For example, clinical symptoms of allopregnanolone withdrawal may occur in women post-partum and/or during the menstrual cycle. Further, clinical symptoms of allopregnanolone may occur in connection with stress in both women and/or men. Clinical symptoms of androstanediol withdrawal may occur in men especially during seasonal changes and/or in connection to strong emotional experiences, such as military combat. Clinical symptoms of deoxycorticosterone, such THDOC, withdrawal may occur in relation to stress such as acute stress.

For the avoidance of any doubt, as used herein the terms “manifestation of (clinical) symptom(s) of substance withdrawal disorder” and “manifestation of substance withdrawal disorder” refer to the same phenomenon and are used interchangeably. As explained above, said substance withdrawal disorder may manifest differently between individual patients. In other words the manifestation of (clinical) symptom(s) of substance withdrawal disorder may vary between individual patients. The manifestation may be one or several of headache, migraine, seizure(s), depression, anxiety and/or panic.

In one embodiment, there is provided a GABA_{A} receptor modulating steroid antagonist for use in treatment of a substance withdrawal disorder as disclosed herein, wherein said treatment is preventive treatment of a substance withdrawal disorder.

For example, in the case when said substance withdrawal disorder is associated with and/or attributed to withdrawal of allopregnanolone, such as in the case of a menstrual cycle related substance withdrawal disorder or post-partum related substance withdrawal disorder, any one of or several of the clinical symptom(s) related to the substance withdrawal, including but not limited to headache, migraine, seizure(s), depression anxiety and/or panic may occur. The skilled person is familiar with the cyclic production of allopregnanolone during the menstrual cycle. Also, symptoms/manifestations of substance withdrawal disorder may be linked to periods of stress in patients, such as women and/or men. It will be understood that any one of the manifestations of the substance withdrawal disorder may include but is not limited to headache, migraine, seizure(s), depression anxiety and/or panic. For example, in the case when said substance withdrawal disorder is associated with and/or attributed to withdrawal of androstanediol, such as may occur during seasonal changes, any one of the manifestations of the substance withdrawal disorder may include but not be limited to headache, migraine, seizure(s), depression, anxiety and/or panic.

For example, in the case when said substance withdrawal disorder is associated with and/or attributed to withdrawal of endogenous PAM such as in the case of acute stress, any one of the manifestations of the substance withdrawal disorder may include but not be limited to headache, migraine, seizure(s), depression, anxiety and/or panic. One specific example of such symptoms is the so called “weekend headache”, which manifests as a headache and/or migraine, but may also include other symptom(s) such as seizure(s), depression, anxiety and/or panic. For clarity, the above mentioned examples are not to be construed as limiting in any way.

Thus, in one embodiment, said disorder is headache associated with and/or attributed to substance withdrawal. The substance withdrawal may be allopreganolone withdrawal, androstanediol withdrawal and/or deoxycorticosterone (such as THDOC) withdrawal.

In one embodiment, said disorder is headache associated with and/or attributed to allopregnanolone withdrawal.

In one embodiment, said disorder is headache associated with and/or attributed to androstanediol withdrawal.

In one embodiment, said disorder is headache attributed to, deoxycorticosterone withdrawal, such as tetra-hydro-deoxycorticosterone (THDOC) withdrawal.

In another embodiment, said disorder is migraine associated with and/or attributed to substance withdrawal. The substance withdrawal may be allopreganolone withdrawal, androstanediol withdrawal and/or deoxycorticosterone (such as THDOC) withdrawal.

In one embodiment, said disorder is migraine associated with and/or attributed to allopregnanolone withdrawal.

In one embodiment, said disorder is migraine associated with and/or attributed to androstanediol withdrawal.

In one embodiment, said disorder is migraine associated with and/or attributed to deoxycorticosterone withdrawal, such as tetra-hydro-deoxycorticosterone (THDOC) withdrawal.

In yet another embodiment, said disorder is a seizure disorder associated with and/or attributed to substance withdrawal. The substance withdrawal may be allopreganolone withdrawal, androstanediol withdrawal and/or deoxycorticosterone (such as THDOC) withdrawal.

In one embodiment, said disorder is a seizure disorder associated with and/or attributed to allopregnanolone withdrawal.

In one embodiment, said disorder is a seizure disorder associated with and/or attributed to androstanediol withdrawal.

In one embodiment, said disorder is a seizure disorder associated with and/or attributed to deoxycorticosterone withdrawal, such as tetra-hydro-deoxycorticosterone (THDOC) withdrawal.

In one another embodiment, said disorder is an anxiety disorder associated with and/or attributed to substance withdrawal.

In one embodiment, said disorder is an anxiety disorder associated with and/or attributed to allopregnanolone withdrawal.

In one embodiment, said disorder is an anxiety disorder associated with and/or attributed to androstanediol withdrawal.

In one embodiment, said disorder is an anxiety disorder associated with and/or attributed to deoxycorticosterone withdrawal, such as tetra-hydro-deoxycorticosterone (THDOC) withdrawal.

In another embodiment, said disorder is a panic disorder associated with and/or attributed to substance withdrawal. The substance withdrawal may be allopreganolone withdrawal, androstanediol withdrawal and/or deoxycorticosterone (such as THDOC) withdrawal.

In one embodiment, said disorder is a panic disorder associated with and/or attributed to allopregnanolone withdrawal.

In one embodiment, said disorder is a panic disorder associated with and/or attributed to androstanediol withdrawal.

In one embodiment, said disorder is a panic disorder associated with and/or attributed to deoxycorticosterone withdrawal, such as tetra-hydro-deoxycorticosterone (THDOC) withdrawal.

In another embodiment, said disorder is a depression associated with and/or attributed to substance withdrawal. The substance withdrawal may be allopreganolone withdrawal, androstanediol withdrawal and/or deoxycorticosterone (such as THDOC) withdrawal.

In one embodiment, said disorder is a depression associated with and/or attributed to allopregnanolone withdrawal.

In one embodiment, said disorder is a depression associated with and/or attributed to androstanediol withdrawal.

In one embodiment, said disorder is a depression associated with and/or attributed to deoxycorticosterone withdrawal, such as tetra-hydro-deoxycorticosterone (THDOC) withdrawal.

For the sake of clarity, it will be appreciated that substance withdrawal disorder may manifest as one of several of headache, migraine, seizure(s), depression, anxiety and/or panic. Said headache, migraine, seizure(s), depression, anxiety and/or panic are by no means to be interpreted as exclusive and two or more thereof may occur simultaneously and/or consecutively as manifestations of said substance withdrawal disorder.

In particular, the present inventors have found that 3beta-hydroxy-5alpha-pregnan-20-one, or a pharmaceutically acceptable salt thereof, or 3beta-fluor-5alpha-pregnan-20-one, or a pharmaceutically acceptable salt thereof, may be used in treatment of a substance withdrawal disorder.

Thus, in one embodiment, said GABA_{A} receptor modulating steroid antagonist (GAMSA) described herein may be 3beta-hydroxy-5alpha-pregnan-20-one, or a pharmaceutically acceptable salt thereof.

In one embodiment, said GABA_{A} receptor modulating steroid antagonist is 3beta-fluor-5alpha-pregnan-20-one, or a pharmaceutically acceptable salt thereof.

The present disclosure also provides the GABA_{A} receptor modulating steroid antagonist in combination with an additional active agent. The additional agent may be one or more compounds as described herein.

Thus, in one embodiment, said GABA_{A} receptor modulating steroid antagonist is provided in combination with one or more compounds selected from the group consisting of a nonsteroidal anti-inflammatory drug, a menstrual cycle regulating compound, a triptan, an NSAID-triptan combination, a non-opioid combination analgesic, an anti-emetic, an antiepileptic drug, a beta blocker, and an antidepressant. In one embodiment, said GABA_{A} receptor modulating steroid antagonist is provided in combination with one or more compounds selected from the group consisting of a nonsteroidal anti-inflammatory drug, a menstrual cycle regulating compound, a triptan, an NSAID-triptan combination, a non-opioid combination analgesic, an anti-emetic, an antiepileptic drug, a beta blocker, an antidepressant and/or antibodies against calcitonin gene-related peptide (CGRP) or the receptor to CGRP e.g. Erenumab and Fremanezumab-vfrm.

The combination of the GABA_{A} receptor modulating steroid antagonist and the additional active agent as described herein may be provided as a single composition. Alternatively, the combination may be provided as a kit of parts comprising or consisting of:

(i) a GABA_{A} receptor modulating steroid antagonist as described herein,
(ii) an additional active agent as described herein, and optionally
(iii) instructions for use.

In one embodiment, said GABA_{A} receptor modulating steroid antagonist is provided in combination with one or more compounds selected from the group consisting of acetaminophen, ibuprofen, acetylsalicylic acid, naproxen sodium, diclofenac potassium, almotriptan, eletriptan, frovatriptan, naratriptan, rizatriptan, sumatriptan, and zolmitriptan, dihydroergotamine, non-opioid acetaminophen, caffeine, metoclopramide, domperidone, prochlorperazine, etoprolol, propranolol, onabotulinumtoxin A, amitriptyline, venlafaxine, fluoxetine, topiramate, lamotrigine, gabapentin and carbamazepine. In one embodiment, said GABA_{A} receptor modulating steroid antagonist is provided in combination with one or more compounds selected from the group consisting of acetaminophen, ibuprofen, acetylsalicylic acid, naproxen sodium, diclofenac potassium, almotriptan, eletriptan, frovatriptan, naratriptan, rizatriptan, sumatriptan, zolmitriptan, dihydroergotamine, non-opioid acetaminophen, caffeine, metoclopramide, domperidone, prochlorperazine, etoprolol, propranolol, onabotulinumtoxin A, amitriptyline, venlafaxine, fluoxetine, topiramate, lamotrigine, gabapentin, carbamazepine, Erenumab and Fremanezumab-vfrm.

The present inventors have come to the surprising realization, that the efficiency of treating a substance withdrawal disorder is strongly dependent on initiating the treatment before the manifestation or onset of the symptom(s) of the substance withdrawal disorder (Bäckstrom et al 2005). Therefore, the treatment may be initiated at the time of tolerance development or before onset of tolerance development. Thus, a preventive treatment is therefore disclosed herein as explained above.

The skilled person is familiar with the concept of substance tolerance, which is the pharmacological concept describing a patient's reduced effect of and/or reaction to a substance following prolonged use of a medicine or exposure to an endogenous substance for a prolonged period of time. Increasing the dosage may re-amplify the effects of a substance, however this may accelerate tolerance, further reducing effects of said substance. Substance tolerance is indicative of substance use and may be associated with substance dependence or addiction.

Thus, as used herein, the term “tolerance development” refers to a decrease in sensitivity to a drug or substance, for example endogenous substance, that occurs after multiple exposures to the drug or substance, for example endogenous substance, or long production of the drug or substance, for example endogenous substance. The effect of a certain substance/drug can change through repetitive or constant input of that substance. In other words, the same dosage does may not have the same clinical effect and the dose must be increased in order for the initial effect to be reached.

As used herein, the term “time point of onset of tolerance development” refers to the time point when tolerance starts to develop. The time point of manifest tolerance development is the time point, such as the first time point, when a discontinuation of exposure to the substance leads to the manifestation of the substance withdrawal disorder.

To clarify, at the time point of onset of tolerance development a blockade or inhibition of the effect of the substance to which tolerance has started to develop, can hinder the manifestation of the substance withdrawal disorder, in other words said blockade or inhibition of the effect of the substance to which tolerance has started to develop thus becomes a prophylactic treatment against said withdrawal disorder.

The treatment described herein may be initiated before manifestation of the substance withdrawal disorder. Thus, in one embodiment there is provided a GABA_{A} receptor modulating steroid antagonist for use as described herein, wherein said treatment takes place before the manifestation said substance withdrawal disorder.

In one embodiment, there is provided a GABA_{A} receptor modulating steroid antagonist for use in treatment of a substance withdrawal disorder, wherein said treatment is initiated no later than at the time point of onset of tolerance development to the substance in the patient.

In one embodiment, said treatment is initiated prior to the time point of onset of tolerance development to the substance in the patient.

The skilled person will appreciate that the substance referred to in a substance withdrawal disorder in the phrase “substance withdrawal disorder” and phrase “onset of tolerance development to the substance” is the same substance or substantially the same substance.

Hence, the treatment may be initiated before the manifestation such as the expected manifestation, of the substance withdrawal disorder.

As used herein, the expression “before/prior to the expected manifestation of (clinical) symptom(s)” or “before/prior to the expected manifestation substance withdrawal disorder” refers to the time point before the disorder would normally occur in a patient who is not undergoing treatment with GABA_{A} receptor modulating steroid antagonist. It is to be understood that the time of initiation of treatment in relation to the manifestation, such as the expected manifestation, of substance withdrawal disorder may vary between different patients. It will be understood that the initiation of the treatment for a specific patient should be related to the situation when said patient was not undergoing treatment (for example based on medical history) alternatively the time of manifestation, such as expected manifestation, of the substance withdrawal disorder may be based on an average time calculated based on data from other patients. Additionally, the time point for onset is also dependent on the circumstances of the tolerance development.

The time point is not an exact fixed time point for each patient, but rather is to be interpreted as a time range, and is at least to be interpreted as at most +/−25%, such as at most +/−20%, such as at most +/−15%, such as at most +/−10%, in relation to the given time point, provided that said time point is not 0 days or the range does not encompass 0 days and wherein said at most +/−25% such as at most +/−20%, such as at most +/−15%, such as at most +/−10%, of said time point is not less than 1 day.

The examples given below are calculated with a time range of +/−25%, the skilled person will appreciate that the same calculation principle applies for time ranges +/−20%, +/−15% and +/−10%. For example if the expected manifestation of the symptom(s) of the substance withdrawal disorder are expected in 8 days in the patient, said time point is 8 days and the correct interpretation of the time range is 8 +/−2 days, in other words 10-6 days before the manifestation of symptom(s). For clarity, if said +/−25% is a fraction of a day, it is to be interpreted as a full day. For example, if said time point is 9 days, the correct interpretation of the time range is 9 +/−3 days, in other words 12-6 days. In the interest of clarity, for example in the instance where the time point is 1 day, the correct interpretation of the time range is 1+/−1 days, in other words 1-2 days as the time range as defined herein does not encompass 0 days. As another example in the interest of clarity, for example if the time point is given as 2 weeks, the correct interpretation thereof is 14+/−4 days, in other words 18-10 days. In the instance where the time point is 1 week, the correct interpretation is 9-5 days. For the interest of clarity, for the calculations used herein, a month is considered to correspond to 30 days, independent of the identity of the calendar month. Kindly note that the time range as used herein is to be interpreted in days independently of if the time point is given in weeks or months or years.

Thus in one embodiment, there is provided a GABA_{A} receptor modulating steroid antagonist for use as disclosed herein, wherein said treatment is initiated at a time point prior to the manifestation, such as the expected manifestation, of said withdrawal disorder in a patient who is not undergoing treatment with GABA_{A} receptor modulating steroid antagonist.

In one embodiment, said time point is at least about 1 day, such as at least about 2 days, such as at least about 3 days, at least about 4 day, such as at least about 5 days, such as at least about 6 days, such as at least about 7 days, such as at least about 8 days, such as at least about 9 days, such as at least about 10 days, such as at least about 11 days, such as at least about 12 days, such as at least about 13 days, such as at least about 14 days, such as at least about 15 days, such as at least about 16 days, such as at least about 17 days, such as at least about 18 days, such as at least about 19 days, such as at least about 20 days, such as at least about 21 days, such as at least about 22 days, such as at least about 24 days, such as at least about 25 days, such as at least about 26 days, at least about 27 days, such as at least about 28 days, prior to the expected manifestation of said substance withdrawal disorder.

Thus, in one embodiment as disclosed herein, there is provided a GABA_{A} receptor modulating steroid antagonist for use in treatment of a substance withdrawal disorder, wherein said treatment is initiated at a time point at least from about 2 to about 3 days before the manifestation, such as the expected manifestation, of the substance withdrawal disorder.

In one embodiment, said treatment is initiated at a time point of from about 2 to about 21 days, such as from about 2 to about 20 days, such as from about 2 to about 19 days, such as from about 2 to about 18 days, such as from about 2 to about 17 days, such as from about 2 to about 16 days, such as from about 2 to about 15 days, such as from about 2 to about 14 days, such as from about 2 to about 13 days, such as from about 2 to about 12 days, such as from about 2 to about 11 days, such as from about 2 to about 10 days, such as from about 2 to about 9 days, such as from about 2 to about 8 days, such as from about 2 to about 7 days, such as from about 2 to about 6 days, such as from about 2 to about 5 days, such as from about 2 to about 4 days, such as from about 2 to about 3 days prior to the expected manifestation of the substance withdrawal disorder.

In one embodiment, said time point encompasses the time range of +/−25%, such as the time range +/−20%, such as the time range +/−15%, such as the time range +/−10%. For the avoidance of any doubt, the time range is applicable to any one of above mentioned time points.

In some embodiments, it may be beneficial to initiate the treatment at least about 1, 2 or 3 weeks prior to the expected manifestation of the symptoms of the substance withdrawal disorder.

For example, in the case of a menstrual cycle related substance withdrawal disorder, such as a substance withdrawal disorder associated with and/or attributed to allopregnanolone withdrawal, the expected manifestation of symptom(s) in a patient who is not undergoing treatment with GABA_{A} receptor modulating steroid antagonist coincide with onset of the menstrual bleeding and it is envisioned that it may be beneficial to initiate the treatment from about 10 to about 14 days prior to the expected manifestation of the substance withdrawal disorder. Examples of menstrual cycle related substance withdrawal disorder include headache and migraine, such as menstrual migraine.

In the case of pregnancy, it is envisioned that it would be beneficial to initiate the treatment about 21 days prior to the expected manifestation of the post-partum substance withdrawal disorder. In particular, said substance withdrawal disorder may be associated with and/or attributed to allopregnanolone withdrawal.

Thus in one embodiment, there is provided a GABA_{A} receptor modulating steroid antagonist for use in treatment of a substance withdrawal disorder associated with and/or attributed to allopregnanolone withdrawal, wherein said treatment is initiated from about 1 to about 3 weeks, such as about 1, 2 or 3 weeks, prior to the expected manifestation of the substance withdrawal disorder.

In one embodiment, said treatment is initiated at a time point of from about 10 to about 16 days, such as from about 10 to about 14 days, such as from about 11 to about 13 days, such as from about 12 days prior to the expected manifestation of the substance withdrawal disorder. In one embodiment, said treatment is initiated at a time point of about 12+/−3 days, such as about 12+/−2 days, such as about 12+/−1 day, such as about 12 days prior to the expected manifestation of the substance withdrawal disorder. In one embodiment, said substance withdrawal disorder is a menstrual cycle related substance withdrawal disorder.

In one embodiment, said expected manifestation of the substance withdrawal disorder coincides with onset of menstrual bleeding.

In one embodiment, said treatment is initiated at a time point of about 3 weeks +/−1 week, prior to the expected manifestation of the substance withdrawal disorder. In one embodiment, wherein said substance withdrawal disorder is a post-partum related substance withdrawal disorder. In one embodiment, said expected manifestation of the substance withdrawal disorder is post-partum, such as within about 1 month after child birth.

In one embodiment, said substance withdrawal disorder is headache, migraine, seizure(s), depression, anxiety and/or panic, associated with and/or attributed to allopregnanolone withdrawal, such as headache and/or migraine associated with and/or attributed to allopregnanolone withdrawal.

In some embodiments, it may be beneficial to initiate the treatment at least about 1, 2 or 3 days prior to the expected manifestation of the substance withdrawal disorder. In the case of stress related head ache also known as “week end headache”, it is envisioned that it may be beneficial to initiate the treatment prior to the reduction of stress. For example, in the case of “week end headache” it is envisioned that treatment is initiated about 2 to about 3 days prior to the expected manifestation of symptoms. For example, if symptoms are expected to occur on Saturday, treatment may be initiated between Tuesday and Friday morning +/−1 day, such as for example Wednesday evening +/−1 day.

Thus in one embodiment, there is provided a GABA_{A} receptor modulating steroid antagonist for use in treatment of a substance withdrawal disorder associated with and/or attributed to allopregnanolone and/or THDOC withdrawal, wherein said treatment is initiated at a time point of from about 1 to about 3 days, such as about 1, 2 or 3 days, prior to the expected manifestation of the symptoms of the substance withdrawal disorder.

In one embodiment, said substance withdrawal disorder is headache, migraine, seizure(s), depression, anxiety and/or panic associated with and/or attributed to allopregnanolone and/or THDOC withdrawal, such as headache or migraine associated with and/or attributed to allopregnanolone and/or THDOC withdrawal. In one embodiment, said substance withdrawal disorder is a stress related substance withdrawal disorder.

In another embodiment, said time point is at least about 1 week, such as at least about 2 weeks, such as at least about 3 weeks, such as at least about 4 weeks, such as at least about 5 weeks, such as at least about 6 weeks, such as at least about 7 weeks, such as at least about 8 weeks.

In one embodiment, said time point encompasses the time range of +/−25%, such as the time range +/−20%, such as the time range +/−15%, such as the time range +/−10%.

In some embodiments, the treatment may be initiated from about 6 to about 3, from about 4 to about 3, or from about 4 to about 2 months prior to the expected manifestation of the symptoms of the substance withdrawal disorder. In the case of androstanediol related headache which commonly manifests during the summer, it is envisioned that it may be beneficial to initiate the treatment during late spring/early summer. For example, said treatment may be initiated in March-June +/−1 month, for example in early May +/−1 month. Thus, in one embodiment, there is provided a GABA_{A} receptor modulating steroid antagonist for use in treatment of a substance withdrawal disorder associated with and/or attributed to androstanediol withdrawal, wherein said treatment is initiated at a time point of from about 1 to about 6 months, such as 6, 5, 4, 3, 2 or 1 month, prior to the expected manifestation of the symptoms of the substance withdrawal disorder.

In another embodiment, said time point is at least about 1 month, such as at least about 2 months, such as at least about 3 months, such as at least about 4 months. In one embodiment, said time point encompasses the time range of +/−25%, such as the time range +/−20%, such as the time range +/−15%, such as the time range +/−10%.

In one embodiment, said substance withdrawal disorder is headache, migraine, seizure(s), depression, anxiety and/or panic, associated with and/or attributed to androstanediol withdrawal. In one embodiment, said substance withdrawal disorder is headache and/or migraine associated with and/or attributed to androstanediol withdrawal. In one embodiment, said substance withdrawal disorder is a season related substance withdrawal disorder.

The GABA_{A} receptor modulating steroid antagonist described herein may form part of a pharmaceutical composition. In one embodiment, there is provided a pharmaceutical composition comprising a GABA_{A} receptor modulating steroid antagonist, for use as described herein, together with one or more pharmaceutically acceptable carriers, excipients and/or diluents. The GABA_{A} receptor modulating steroid antagonist may be 3beta-hydroxy-5alpha-pregnan-20-one and/or 3beta-fluor-5alpha-pregnan-20-one. Further, the GABA_{A} receptor modulating steroid antagonist may be a pharmaceutically acceptable salt of 3beta-hydroxy-5alpha-pregnan-20-one and/or 3beta-fluor-5alpha-pregnan-20-one.

In one related aspect of the present disclosure, there is provided a method of treating, preventing and/or alleviating a substance withdrawal disorder, comprising administering a pharmaceutically effective amount of a GABA_{A} receptor modulating steroid antagonist as described herein, to a patient in need thereof. Embodiments of this aspect equal the embodiments described above for the aspect related to GABA_{A} receptor modulating steroid antagonists for use in treatment of a substance withdrawal disorder and will not be repeated here for the sake of brevity.

In another aspect of the present disclosure, there is provided a use of a GABA_{A} receptor modulating steroid antagonist as described herein in the manufacture of a medicament for the treatment of a substance withdrawal disorder as described herein, such as for the preventive treatment a substance withdrawal disorder. In one embodiment, there is provided a use of a GABA_{A} receptor modulating steroid antagonist in the manufacture of a medicament, wherein said substance withdrawal disorder is caused by withdrawal of a positive GABA_{A} receptor modulating steroid. Embodiments of this aspect equal the embodiments described above for the first aspect related to GABA_{A} receptor modulating steroid antagonists for use in treatment of a substance withdrawal disorder and will not be repeated here for the sake of brevity.

The phrase “GABA_{A} receptor modulating steroid antagonists” is meant to define compounds that only antagonize or block the action of positive GABA_{A} receptor modulating steroids. These compounds have no antagonistic effect on GABA's effect on the GABA_{A} receptor and are thus not negative modulators or inverse agonists.

“GABA_{A} receptor modulating steroid antagonists” include, but are not limited to, 3beta-hydroxy-5alpha-pregnan-20-one and 3beta-fluor-5alpha-pregnan-20-one. Further GABA_{A} receptor modulating steroid antagonists are inter alia disclosed in WO 2008/063128, which are incorporated herein by reference.

The clinical indication “substance withdrawal disorder” includes, but is not limited to, headache associated with and/or attributed to substance withdrawal; headache associated with and/or attributed to allopregnanolone withdrawal; headache associated with and/or attributed to androstanediol withdrawal; headache associated with and/or attributed to deoxycorticosterone withdrawal, such as tetra-hydro-deoxycorticosterone (THDOC) withdrawal; migraine associated with and/or attributed to substance withdrawal; migraine associated with and/or attributed to allopregnanolone withdrawal; migraine associated with and/or attributed to androstanediol withdrawal; migraine associated with and/or attributed to deoxycorticosterone withdrawal, such as tetra-hydro-deoxycorticosterone (THDOC) withdrawal; a seizure disorder associated with and/or attributed to substance withdrawal; a seizure disorder associated with and/or attributed to allopregnanolone withdrawal; and a seizure disorder associated with and/or attributed to androstanediol withdrawal; seizure disorder associated with and/or attributed to deoxycorticosterone withdrawal, such as tetra-hydro-deoxycorticosterone (THDOC) withdrawal; a depression associated with and/or attributed to substance withdrawal; a depression associated with and/or attributed to allopregnanolone withdrawal; and a depression associated with and/or attributed to androstanediol withdrawal; depression associated with and/or attributed to deoxycorticosterone withdrawal, such as tetra-hydro-deoxycorticosterone (THDOC) withdrawal; anxiety associated with and/or attributed to substance withdrawal; anxiety associated with and/or attributed to allopregnanolone withdrawal; anxiety associated with and/or attributed to androstanediol withdrawal; anxiety associated with and/or attributed to deoxycorticosterone withdrawal, such as tetra-hydro-deoxycorticosterone (THDOC) withdrawal; panic disorders associated with and/or attributed to substance withdrawal; panic disorders associated with and/or attributed to allopregnanolone withdrawal; panic disorders associated with and/or attributed to androstanediol withdrawal; and panic disorders associated with and/or attributed to deoxycorticosterone withdrawal, such as tetra-hydro-deoxycorticosterone (THDOC) withdrawal.

With the phrase “allopregnanolone withdrawal” is meant a disorder, syndrome or set of symptoms arriving at the end of exposure to (in other words withdrawal of) allopregnanolone after a period of allopregnanolone exposure.

With the phrase “androstanediol withdrawal” is meant a disorder, syndrome or set of symptoms arriving at the end of exposure to (in other words withdrawal of) androstanediol after a period of androstanediol exposure.

With the phrase “deoxycorticosterone withdrawal, such as tetra-hydro-deoxycorticosterone (THDOC) withdrawal” is meant a disorder, syndrome and/or set of symptoms arriving at the end of exposure to (in other words withdrawal of) deoxycorticosterone withdrawal, such as tetra-hydro-deoxycorticosterone (THDOC) after a longer period of deoxycorticosterone withdrawal, such as tetra-hydro-deoxycorticosterone (THDOC) exposure.

With the phrase “headache” is meant the symptom of pain anywhere in the region of the head or neck. Headache includes, but is not limited to, substance withdrawal headache general headache (sharp, or diffuse pains), tension-type headaches, and cluster headaches. The International Headache Society's International Classification of Headache Disorders (ICHD), which published the second edition in 2004, 2013, 2016.

As used herein, the term “weekend headache” refers to a headache that follows after a period of stress has ended, such for example when work-related stress is interrupted during weekends or vacation. For clarity, weekend headache may manifest as symptoms of headache and/or migraine. The skilled person appreciates that the term “weekend headache” is not to be interpreted to relate to the days of the week and may occur at any time point when a period of stress has ended.

With the phrase “migraine” is meant a primary headache disorder characterized by recurrent headaches that are moderate to severe. Often the headache affects half the head, is pulsating, and lasts from 2-72 hours (ICHD). Migraine includes, but is not limited to substance withdrawal induced migraine; migraine with associated symptoms like nausea, vomiting, sensitivity to light, sensitivity to sound, sensitivity to smell and/or an aura (which is a short period of visual disturbance).

With the phrase “seizure disorder” or “seizure(s)” is meant episodes of vigorous shaking and unconsciousness that can vary from brief to long periods. Seizure disorders include, but are not limited to substance withdrawal induced seizure(s), epilepsy, generalized seizure(s) affecting both hemispheres of the brain and/or focal seizure(s) which affect part of the brain.

With the phrase “depression” is meant episodes of either (1) depressed mood or (2) loss of interest or pleasure that can vary from short to long periods. The individual must be experiencing five or more symptoms during the same 2-week period according to description in Diagnostic and Statistical Manual of Mental Disorders (DSM), the DSM-5. Depression includes, but is not limited to substance withdrawal induced depression, stress induced depression, seasonal depression.

With the phrase “panic disorder” is meant a disorder characterized by reoccurring, unexpected panic attacks. Panic attacks are sudden periods of intense fear that may include heart palpitations, sweating, shaking, shortness of breath, numbness, or a bad feeling. Panic disorder includes, but is not limited to, substance withdrawal induced panic attacks, familiar panic disorder stress-induced or smoke-induced panic attacks.

With the phrase “anxiety disorder” is meant a group of mental disorders characterized by significant feelings of anxiety and fear. Anxiety is a worry about future events and fear is a reaction to current events. These feelings may cause physical symptoms, such as a fast heart rate and shakiness. Anxiety disorder includes, but is not limited to, substance withdrawal induced anxiety attacks generalized anxiety disorder, specific phobia, social anxiety disorder, separation anxiety disorder, agoraphobia, panic disorder, and selective mutism.

It is to be understood that the terminology employed herein is used for the purpose of describing particular embodiments only and is not intended to be limiting, since the scope of the present invention will be limited only by the appended claims and equivalents thereof.

In particular, it is noted that, as used in this specification and the appended claims, the singular forms “a”, “an”, and “the” also include plural referents unless the context clearly dictates otherwise. Also, as used herein, the term “at least one of” is to be understood as one or more.

The terms “antagonizing and blocking” is meant to define an effect where in this case the 3alpha-hydroxy-5alpha/beta-steroids are prevented from acting on the GABA_{A} receptor. It is understood that “blocking” is an entirely different effect than that meant by “modulation” or “repression” or similar terms, which suggest that an action is still taking place, but to a lesser extent or at a slower rate.

The term “pharmaceutical composition” is used in its widest sense, encompassing all pharmaceutically applicable compositions containing at least one active substance, and optional carriers, adjuvants, constituents etc. The term “pharmaceutical composition” also encompasses a composition comprising the active substance in the form of derivate or a prodrug, such as a pharmaceutically acceptable salt, sulphate and/or ester of said active substance. The active substance may be a GABA_{A} receptor modulating steroid antagonist as described herein. The manufacture of pharmaceutical compositions for different routes of administration falls within the capabilities of a person skilled in galenical chemistry.

The terms “administration” and “mode of administration” as well as “route of administration” are also used in their widest sense. The pharmaceutical composition of the present disclosure may be administered in a number of ways depending largely on whether a local, topical or systemic mode of administration is most appropriate for the condition to be treated. These different modes of administration are for example topical, local, oral, parenteral and/or pulmonary administration. The preparation of such compositions and formulations is generally known to those skilled in the pharmaceutical and formulation arts and may be applied to the formulation of the composition of the present disclosure.

The term “UC1010” equals to 3beta-hydroxy-5alpha-pregnan-20-one, wherever used.

The term “UC2016” equals to 3beta-fluor-5alpha-pregnan-20-one, wherever used.

With the phrase “GAMSA” is meant compounds that only antagonize the action of positive GABA_{A} receptor modulating steroids. When such compounds only antagonize or block the action of positive GABA_{A} receptor modulating steroids, they have a “GAMSA effect”. The abbreviation “GAMSA” stands for GABA_{A} receptor modulating steroid antagonist.

The term “LoR” equals to loss of righting reflex, wherever used.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE DRAWINGS

FIG. 1 illustrates that 3beta-hydroxy-5alpha-pregnan-20-one (UC1010) antagonized the THDOC effect. Further details are set out in Example 1.

FIG. 2 illustrates that 1 μM 3beta-fluor-5alpha-pregnan-20-one (UC2016) reduced the 30 μM GABA mediated current response in presence of 200 nM THDOC and slightly in the absence of THDOC. Control, 30 μM GABA ±200 nM THDOC, was set to 0. Further details are set out in Example 2.

FIG. 3 provides an outline of the study paradigm including estrus cycle phase determination and investigation of tolerance symptoms using resident/intruder (R/I) tests. Further details are set out in Example 3.

FIG. 4 illustrates baseline tolerance symptoms of two estrus cycles. Further details are set out in Example 3.

FIG. 5 illustrates tolerance symptoms and normal social behavior during 3beta-hydroxy-5alpha-pregnan-20-one treatment compared to mean baseline. Further details are set out in Example 3.

FIG. 6 shows the chemical structures of (A) 3beta-hydroxy-5alpha-pregnan-20-one, (B) 3beta-fluor-5alpha-pregnan-20-one, (C) allopregnanolone, (D) androstanediol, (E) deoxycorticosterone and (F) tetra-hydro-deoxycorticosterone (THDOC).

### EXAMPLE 1

Testing of GABA_{A} Receptor Effects of 3Beta-Hydroxy-5Alpha-Pregnan-20-One on Human Alpha1,Beta,Gamma and Alpha4,Beta,Delta GABA_{A} Receptor Subtype.

Aim: To investigate the effect of 3beta-hydroxy-5alpha-pregnan-20-one on

1) GABA_{A} receptor function in the absence and presence of GABA and
2) in absence and in presence of the positive GABA_{A} receptor modulating steroids Tetrahydrodeoxycorticosterone (THDOC) and 3alpha-hydroxy-5alpha-androstan-17-ol (3alpha-OH-adiol) by the Dynaflow™ system on HEK-293 cells. In these tests the protocol was optimized to be similar to the physiological conditions in the synaptic cleft.

Cell culture: HEK-293 cells, permanently transfected with the human alpha1,beta,gamma and alpha4,beta,delta GABA_{A} receptor subtypes, were seeded at a density of 3×10⁴/25 cm² in a culture flask. The transfected cells were used for patch-clamp experiments 3 days after seeding. When using the cells for patch-clamp experiments the cells were washed twice with oxygen-bubbled extra cellular (EC) solution (see below). About 5 mL EC was then added and the cells were kept in the incubator for about 15 minutes. After 15 minutes the cells came loose from the bottom of the flask and were separated by carefully sucking a couple of times with a Pasteur pipette.

Dynaflow™ system with Resolve chip was used for patch-clamp experiments: The electrophysiological recordings from HEK-293 cells were performed under voltage-clamp conditions using patch-clamp technique and the Dynaflow™ system (Dynaflow Pro II Platform Zeiss Axiovert 25; Cellectricon AB, Sweden) with Resolve chips as application system.

Patch pipettes were pulled from borosilicate glass and polished to a resistance 2-5 MΩ when filled with suitable intracellular solution (IC) (pH was adjusted to 7.2) and immersed in bath solution (extracellular, EC) (pH is adjusted to 7.4).

The recordings were made using an Axopatch 200B amplifier, a Digidata 1322A (Axon instruments, Foster city, USA). Data were analysed using the pCLAMP software sampled at 10 kHz, filtered at 2-10 kHz and analyzed with Clampfit (versions 9.0, both from Axon instruments, Foster city, USA). No higher series resistance than 20 MΩ between pipette and cell membrane was accepted. No series resistance compensation was used. The stability of series resistance was monitored repeatedly from the time course of capacitative transients during the experiments. The measured liquid-junction potential between EC and IC was subtracted in all data presented. All experiments were performed at room temperature (21-23° C.).

Steroids and GABA: GABA was dissolved in EC-solution by ultra sound for about 40 minutes to the concentration of 10 mM in room temperature. All steroids were dissolved to the concentration of 6 mM in DMSO. The DMSO concentration was 0.1% in all end-solutions, including the wash solution (EC) and the solution with GABA alone. End solutions were the solutions added into the wells of the chip.

Electrophysiology: After compensating for the liquid junction potential a steady holding potential of −17 mV was used in all experiments. In physiological conditions the HEK-293 had a resting potential at −40 mV and a low concentration of chloride ions inside the cell. By using the holding potential of −17 mV and the intracellular solution with low chloride ion concentration the chloride ions flux into the cell when the receptors were activated.

### Protocol

GABA applications: By using the Dynaflow equipment it was possible to study transfected HEK-293 during almost physiological conditions. The Dynaflow system allowed application of solutions for as short as 40 ms up to minutes in time. Physiologically, in the synaptic cleft, GABA was released in mM range for about 2 milliseconds (ms). In these experiments we apply GABA ±steroid for 40 ms. It was found that in almost all cells, the first GABA application gave a smaller response than the second GABA application. There was no difference in response between the second and the third GABA application. Therefore, the first GABA application was always repeated twice and the second response was used in the analysis.

Washout: GABA is quite soluble in water and easy to washout from the receptor. The washout time was set to 1 minute after application with GABA solely. Steroids on the other hand were difficult to dissolve in water and also difficult to washout from the receptor. In the experiments, THDOC and 3alpha-OH-adiol were used as the positive GABA_{A} receptor modulating steroids. With 2 minutes washout time, 200 nM THDOC and 3alpha-OH-adiol was completely washed out as shown by neither an accumulative nor a desensitization effect.

Incubation: To see the effect of the steroids and to achieve stable results it was found that the steroids had to be incubated on the receptor before application of GABA. Different incubation times were studied to achieve the optimal time for attaining stable results and minimize the washout time. Incubation time of 20 seconds showed to be the optimal time for washout time of 2 minutes.

Experiments with Application of 3Beta-Hydroxy-5Alpha-Pregnan-20-One on the GABA_{A} Receptor Subtype Alpha1,Beta2,Gamma2 and GABA_{A} Receptor Subtype Alpha4,Beta,Delta

Experiments with application on the GABA_{A} receptor subtype alpha1,beta2,gamma2 are (a) the inhibitory effect of 3beta-hydroxy-5alpha-pregnan-20-one on the effect of GABA+3alpha-hydroxy-5alpha-pregnan-21-one (THDOC) application at steady state (phasic effect), and (b) effect of 3beta-hydroxy-5alpha-pregnan-20-one on THDOC induced tonic effect (baseline shift). (c) Steroid effect without GABA, steady state and steroid induced baseline shift (without THDOC and GABA), this tests the own effect of 3beta-hydroxy-5alpha-pregnan-20-one on the GABA_{A} receptor subtype alpha1,beta2,gamma2.

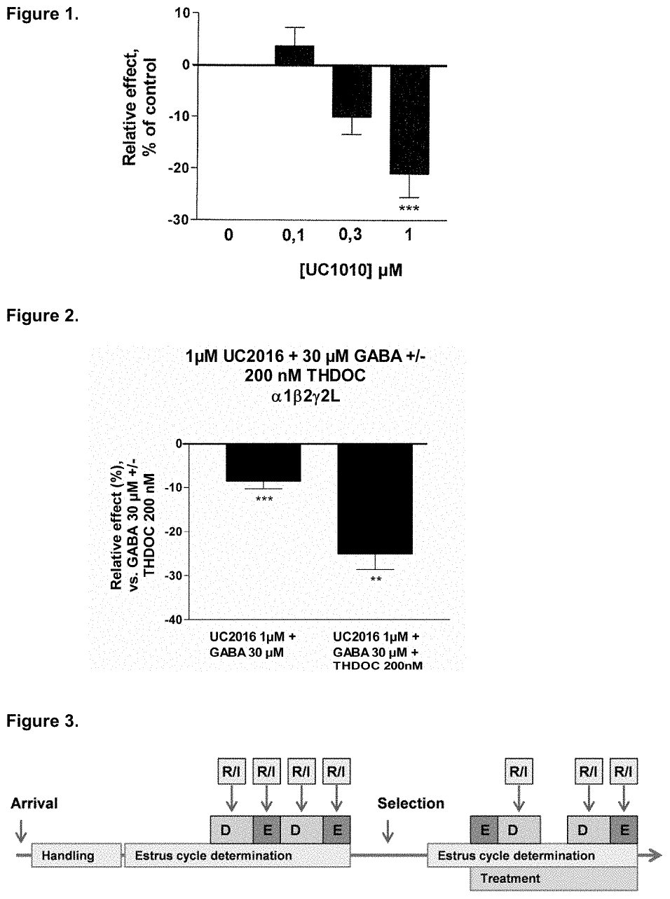

Results: a) In the presence of 30 μM GABA+200 nM THDOC, a single dose of 1 μM 3beta-hydroxy-5alpha-pregnan-20-one antagonized the THDOC enhanced effect by −22.3±5.3% (p<0.001, n=6). In a separate concentration-dependent experiment, 3beta-hydroxy-5alpha-pregnan-20-one was tested in the concentration interval 0.1-1 μM in the presence of 200 nM THDOC and 30 μM GABA (FIG. 1 and table 1). Results show that 3beta-hydroxy-5alpha-pregnan-20-one antagonized the THDOC effect with max −21±4.6% (p<0.001; n=10) which was reached at 1 μM 3beta-hydroxy-5alpha-pregnan-20-one confirming the first single dosage 1 μM experiment.

b) THDOC did not induce any baseline shift in alpha1,beta2,gamma2 receptor subtype and 3beta-hydroxy-5alpha-pregnan-20-one could therefore not be tested (data not shown)
c) The effect of 1 μM 3beta-hydroxy-5alpha-pregnan-20-one (UC1010) in the presence of 30 μM GABA did not significantly antagonize or enhance GABA compared to GABA alone (+10.3±7.8%, NS, n=9, Table 1).

TABLE 1
Summary of the results with 3beta-hydroxy-5alpha-pregnan-
20-one (UC1010) at the receptor alpha1, beta2, gamma2.

| | Iₘₐₓ/Eₘₐₓ | | | |
|---|---|---|---|---|
| Test | Mean ± SEM % | | IC₅₀/EC₅₀ | |
| 1-1000 μM GABA | 2600 | pA | 3.6 | μM |

| | | | |
|---|---|---|---|
| 0.03-1 μM THDOC + 30 μM GABA | +115% | 70 | nM |

| | | | |
|---|---|---|---|
| 1 μM UC1010 + 30 μM GABA | +10.3 ± 7.8% | (N.S, n = 9) | — |
| 1 μM UC1010 + 30 μM GABA + | −21 ± 4.6% | (P < 0.001, n = 10) | — |
| 200 nM THDOC | | | |

| | | | |
|---|---|---|---|
| 0.1-1 μM UC1010 + 30 μM GABA + | −21% | 300 | nM |
| 200 nM THDOC | | | |

The results of application of 3beta-hydroxy-5alpha-pregnan-20-one on a GABA_{A} receptor subtype alpha4,beta,delta is shown in Table 2: a) GABA application, Steady state; b) the inhibitory effect of 3beta-hydroxy-5alpha-pregnan-20-one on the effect of GABA+3alpha-hydroxy-5alpha-pregnan-21-one (THDOC) application on a GABA_{A} receptor subtype alpha4,beta,delta at steady state; c) effect on THDOC induced baseline shift, steroid effect without GABA. The THDOC induces a baseline shift (without THDOC and GABA) in alpha4,beta delta GABA_{A} receptor subtypes (Data not shown).

TABLE 2
Effect of 1 μM 3beta-hydroxy-5alpha-pregnan-20-one
(UC1010) on 3 μM GABA, on THDOC ± 3 μM GABA
at alpha4, beta3, delta receptor.

| The effect of 1 μM UC1010 on | | Mean ± SEM % (P; N) |
|---|---|---|
| a) | 3 μM GABA response (%) | −6.7 ± 3.3% |
| Control | | (N = 11, NS) |
| b) | 0.1 μM THDOC + 3 μM GABA (%) | −26 ± 2.4% |
| | | (N = 13, P = 0.001) |
| c) | 0.1 μM THDOC alone baseline (%) | −43 ± 3.5% |
| | | (N = 11, P = 0.003) |

This surprising result is clearly different from alpha1, beta2, gamma2 results above and surprisingly different from the result in WO 99/45931.

Maximal modulatory effect of 1 μM 3beta-hydroxy-5alpha-pregnan-20-one in presence of 30 μM GABA alone was not significant (10.3±7.8%, NS, n=9). This shows that 1 μM 3beta-hydroxy-5alpha-pregnan-20-one had no effect of GABA itself, therefore no further studies with 3beta-hydroxy-5alpha-pregnan-20-one and GABA alone were performed.

Table 3 shows application of the positive GABA_{A} receptor modulator 3alpha-hydroxy-5alpha-androstan-17-ol on a GABA_{A} receptor subtype alpha1,beta2,gamma2L: a) the inhibitory effect of 3beta-hydroxy-5alpha-pregnan-20-one on the effect of GABA+3alpha-hydroxy-5alpha-androstan-17-ol (3alpha-OH-adiol) application at steady state (phasic effect); b) effect on 3alpha-OH-adiol induced tonic effect (baseline shift); and c) Androstanediol had no effect without GABA (Data not shown).

TABLE 3
Effect of 1 μM 3beta-hydroxy-5alpha-pregnan-20-one (UC1010) on 8 μM
androstanediol ± 30 μM GABA at alpha1, beta2, gamma2.

| The effect of 1 μM UC1010 on | Mean ± SEM % (P; N) |
|---|---|
| a) 8 μM Androstanediol + 30 μM GABA | −30 ± 1.7% |
| (%) | (N = 12, P = 0.001) |
| b) 8 μM Androstanediol, baseline shift (%) | −33 ± 7.6% |
| | (N = 8, P = 0.012) |

The result shows that 3beta-hydroxy-5alpha-pregnan-20-one can inhibit the effect of 3alpha-OH-adiol on the alpha1,beta2,gamma2L receptor subtype.

Table 4 shows application on a GABA_{A} receptor subtype alpha4,beta,delta: a) effect on 3alpha-OH-adiol induced baseline shift; b) the inhibitory effect of 3beta-hydroxy-5alpha-pregnan-20-one on the effect of GABA+3alpha-OH-adiol application on a GABA_{A} receptor subtype alpha4,beta,delta at steady state; c) Androstanediol effect without GABA is shown below in Table 5.

TABLE 4
Effect of 1 μM 3beta-hydroxy-5alpha-pregnan-20-one
(UC1010) on 3 μM GABA, on androstanediol +
3 μM GABA at alpha4, beta3, delta receptor subtype.

| The effect of 1 μM UC1010 on | Mean ± SEM % (P; N) |
|---|---|
| a) 3 μM GABA response (%) | −6.7 ± 3.3% |
| | (N = 11, NS) |
| b) 0.6 μM Androstanediol + | −18 ± 2.0% |
| 3 μM GABA (%) | (N = 9, P = 0.008) |
| b) 0.8 μM Androstanediol + | −22 ± 2.6% |
| 3 μM GABA(%) | (N = 6, P = 0.028) |

TABLE 5
Androstanediol (3 μM and 10 μM) significantly induce current
response in absence of GABA at alpha4, beta3, delta receptor.

| Androstanediol (μM) | Mean ± SEM pA |
|---|---|
| Baseline shift | (N; P) |
| 3 | +8 ± 3.6 pA |
| | (N = 11; P = 0.004) |
| 10 | +13 ± 4.8 pA |
| | (N = 12; P = 0.000) |

Androstanediol (3 μM and 10 μM) had a significant but minor effect on current response in absence of GABA compared to baseline, current response at control-solution (Table 5). Androstanediol induced current response was so low that the effect of 1 μM 3beta-hydroxy-5alpha-pregnan-20-one on the baseline shift was not possible to study.

Finally, 1 μM 3beta-hydroxy-5alpha-pregnan-20-one had no effect by itself at the alpha4,beta3,delta GABA_{A} receptor by itself, −0.4±0.3 pA (N=11, NS) compared to current response at control solution (−0.1±0.3 pA; N=10).

### Conclusions

The results show that 1 μM 3beta-hydroxy-5alpha-pregnan-20-one acted as an antagonist to both androstanediol and to THDOC. 3beta-hydroxy-5alpha-pregnan-20-one significantly reduced the THDOC and androstanediol enhanced GABA mediated effect both at alpha1,beta2,gamma2 and at alpha4,beta3,delta GABA_{A} receptor subtypes. Further, 1 μM 3beta-hydroxy-5alpha-pregnan-20-one significantly reduced the THDOC enhanced GABA mediated current response without any effect on GABA at alpha4,beta3,delta GABA_{A} receptor subtype. 1 μM 3beta-hydroxy-5alpha-pregnan-20-one alone had no effect at the alpha4,beta3,delta GABA_{A} receptor subtype.

Androstanediol enhanced the GABA mediated current response at both alpha1,beta2,gamma2 and alpha4,beta3,delta GABA_{A} receptor subtypes. However, the EC₅₀ and Eₘₐₓ differed between the subtypes. At the alpha1,beta2,gamma2 receptor the Androstanediol EC₅₀ was 3.4 μM compared to 0.38 μM on alpha4,beta3,delta. Also, Eₘₐₓ at the alpha1,beta2,gamma2 was 281% compared to Emax=142% at alpha4,beta3,delta. This provides that androstanediol was more efficient but less potent at the alpha1,beta2,gamma2 receptor compared with alpha4,beta3,delta receptor.

THDOC enhanced the GABA mediated current response at alpha4,beta3,delta GABA_{A} receptor, where the EC₅₀ was 47 nM and Eₘₐₓ was 297%. This provides that THDOC was more efficient and more potent at alpha4,beta3,delta GABA_{A} receptor compared to androstanediol.

### EXAMPLE 2. ANALYSIS OF GABA_{A} RECEPTOR EFFECTS OF 3BETA-FLUOR-5ALPHA-PREGNAN-20-ONE (UC2016) ON HUMAN ALPHA1,BETA,GAMMA GABA_{A} RECEPTOR SUBTYPE

The effect of 3beta-fluor-5alpha-pregnan-20-one (UC2016) in presence of THDOC and GABA on human alpha1,beta2,gamma2 Long subunit GABA_{A} receptors expressed in human embryonic kidney cells (HEK-293 cells) were analyzed. The alpha1,beta2,gamma2 Long subunit composition is the most common GABA_{A} receptor, UC2016 was studied under conditions similar to GABAergic transmission at synapses: high concentration of GABA and short time of application.

Study design: Studies were conducted on current response mediated by chloride ion flux through the GABA_{A} receptor. Patch clamp technique combined with rapid applications of the compounds together and without GABA. The same patch-clamp technique was used as described above (Example 1) using the Dynaflow™ application system. THDOC was used as positive GABA_{A} receptor modulating steroid as it has shorter washout time compared to allopregnanolone but works with the same mechanisms on the same GABA_{A} receptor binding site as allopregnanolone.

HEK-293 cells, permanently transfected with cDNA expressing the human alpha1,beta2,gamma2 Long GABA_{A} receptor subtype. The tissue culture and the handling of the cells were as described in Example 1. In the patch-clamp experiments the transfected cells were used minimum two passages after defrosting and 3 days after seeding. The cells were seeded for maximal 25 times. The cells were detached by the enzyme trypsin by washing with DMEM+Glutamax and incubated with 500 μL trypsin 0.25% lx, for 3 minutes. To terminate the reaction, 10 mL KM was added to the flask and solution was taken to a Falcon tube. The cells were centrifuged for 4.5 minutes in 2500 rpm. Thereafter the pellet was suspended in 2-3 mL EC-solution and kept in heat chamber (37° C.) for about 15-30 minutes.

Electrophysiological recordings: Whole cell patch technique was used to record whole-cell currents from HEK-293 cells as described in Example 1. Borocilicate glass pipettes with a resistance of 2-6 MΩ (Haage et al., 2002; Neher, 1992), a steady holding potential of −17 mV was used in all experiments. The cells were added to the chip and kept in EC solution. EC solution with or without steroid and GABA were applied by the Dynaflow™ system. All experiments were performed at room temperature (21-23° C.). The procedure in experiments with the steroid 3beta-fluor-5alpha-pregnan-20-one and/or THDOC, the steroids was: incubation for 20 seconds before application of GABA+steroid. GABA+steroid were applied for 40 ms followed by a washout period for 2 minutes in EC solution. THDOC and 3beta-fluor-5alpha-pregnan-20-one were dissolved in pure DMSO to the concentration of 6 mM. To completely dissolve the steroid, ultra sound treatment was needed. The final DMSO concentration was 0.1% in all end-solutions, including the wash solution (EC) and the solution with GABA alone.

### Results

3beta-fluor-5alpha-pregnan-20-one (UC2016) inhibited the effect of THDOC+GABA in a dose dependent manner with the highest inhibition at 1 μM UC2016 (see Table 6).

TABLE 6
0.1-3 μM 3beta-fluor-5alpha-pregnan-20-one (UC2016)
in presence of 30 μM GABA and 200 nM THDOC.

| [UC2016] + 30 μM GABA + | Mean ± SEM (%) | |
|---|---|---|
| 200 nM THDOC | (N.P) | |
| 0.1 μM UC2016 | −4.7 ± 3.5 | (N = 20; P = NS) |
| 0.3 μM UC2016 | −11.6 ± 5.5 | (N = 32; P = 0.05) |
| 1 μM UC2016 | −25.1 ± 3.5 | (N = 12; P = 0.002) |

3beta-fluor-5alpha-pregnan-20-one (UC2016) slightly reduces the GABA response but has no effect in the absence of GABA (Table 7, FIG. 2).

TABLE 7
Effect of GABA, THDOC and 3beta-fluor-5alpha-pregnan-20-one
(UC2016) alone on chloride flux measured as currency pA.

| | Iₘₐₓ/Eₘₐₓ | IC₅₀/EC₅₀ | |
|---|---|---|---|
| 1-1000 μM GABA | 2600 pA | 3.6 | μM |
| 0.03-1 μM THDOC + | +115% | 70 | nM |
| 30 μM GABA | | | |
| 1 μM UC2016 + | −8.6 ± 1.7% | | |
| 30 μM GABA | (P ≤ 0.001; n = 26) | | |

| | |
|---|---|
| 1 μM UC2016 | UC2016 had no effect in absence of GABA |

### Conclusions

3beta-fluor-5alpha-pregnan-20-one (UC2016) had an antagonistic effect on GABA mediated current response in presence of THDOC. At 0.1 μM, 0.3 μM and 1 μM, UC2016 reduced the effect of 200 nM THDOC in presence of 30 μM GABA and at 0.3 μM and 1 μM, the UC2016 effect was significant. UC2016 had a slight antagonistic effect, similar to vehicle on GABA mediated current response in presence of GABA alone. UC2016 had no effect on baseline shift, in absence of GABA.

### EXAMPLE 3

### Tolerance Development and Withdrawal Disorder can be Hindered by 3Beta-Hydroxy-5Alpha-Pregnan-20-One Treatment.

Experimental set-up: Experiments were carried out in female cycling rats. Rats have steroid variations related to ovulation similar to humans. In relation to the steroid variations, some rats showed signs of steroid related tolerance symptoms. The symptoms are related to the excitability changes in the brain that precedes withdrawal symptoms. Symptoms like migraine attacks and epileptic seizure(s) will arrive after a period of tolerance (Reddy & Rogawski 2009; Slawecki et al 2000, Welch K M. 2005). Symptoms in rats were investigated using a resident/intruder (R/I) test with behavioral analysis at the diestrus and the estrus phases of the estrus cycle (FIG. 3). The symptoms referred to in this Example are behavioral symptoms of withdrawal.

The rats that showed symptoms at baseline were selected for treatment with 3beta-hydroxy-5alpha-pregnan-20-one. The frequency of behavioral symptom expression at diestrus and estrus during treatment were compared to the same behavior expression prior to treatment. The intruder rats were ovariectomized animals of the same age and size as the residents.

Animals: In total, 42 female Wistar rats (Taconics, Lille Skensved, Denmark) were used. At arrival the animals were approximately 9 weeks old and weighed 185-209 g. For identification, the rats were marked on their tails with permanent ink (PorciMark™). The animals were housed in triads at the animal facility where all experiments took place (Umea University, Umea, Sweden). A reversed light/dark cycle was used (12 h/12 h light/dark; lights off at 04:00). Food (standard chow) and water was available ad libitum. The rats were handled daily for two weeks prior to the start of testing. Nine rats were randomly selected as intruder rats and were ovariectomized. The remaining intact rats were tested for estrus cycle-dependent symptomatic behavior. The study protocol was approved by the Regional Ethics Committee of Umea, Sweden. All animal handling and experiments were performed according to Swedish legislation.

Ovariectomy of the intruder rats: Ovariectomy of the intruder rats was carried out by ligation of uterine tubes and removal of ovaries under anesthesia with 2.3% Isoflurane (Baxter Medical AB, Kista, Sweden) in oxygen (O₂ flow rate 0.32 L/min). The surgical suture techniques included the use of surgical glue (Vetbond Tissue Adhesive No 1469 3M) in addition to sutures. Pain relief (Rimadyl Vet, 0.25 mg/100 g) was given daily for one week post-surgery.

Determination of estrus cycle phase: Estrus cycle phase (proestrus, estrus, metestrus and diestrus) of the rats was determined by microscope examination of vaginal smears. The smears were collected daily between 08:00 and 10:00, using a glass pipette with 50-70 μL normal saline (NaCl 0.9%), and stained with 0.2% toluidine blue. However, on the day of R/I test, the vaginal smear of a tested animal was collected immediately after the encounter. In addition, during the 3beta-hydroxy-5alpha-pregnan-20-one treatment, the smears were collected directly in the morning before injections to confirm expected estrus cycle phase. Diestrus (including metestrus) is characterized by the presence of leucocytes in smears. Both nucleated epithelial cells and cornified epithelial cells are present at diestrus but more abundantly at metestrus than at late diestrus. Estrus (including proestrus) is characterized by thick smears free from leucocytes but with abundant presence of nucleated epithelial cells (predominant in proestrus) and cornified epithelial cell (predominant at estrus). Further details are provided in Hubscher et al., 2005.

During the PM of proestrus and during estrus an increased allopregnanolone production occur and this production is withdrawn during diestrus (Ichikawa S et al 1974). In the rat the time between estrus and diestrus is between 20-26 hours. In the present experiment the treatment started during estrus, that is one day before the withdrawal symptom expression and during the increases production of allopregnanolone.

Investigation of symptoms: Symptoms were investigated during an R/I test (as described in Schneider & Popik, 2007). The R/I tests were performed in dim white light during the animals' dark active period (between 10:00 and 14:00) in a room similar, and adjacent, to their housing room. The resident rat remained in its home cage while its cage mates were removed. After approximately 15 minutes' habituation, an intruder was introduced for a period of eight minutes, and the encounter video recorded with a digital video camera (Sony HDR-CX360E) for later analysis. The intruder rats were marked using colorful felt pens for easy identification during video film analysis. R/I tests were performed at diestrus and estrus of two baseline estrus cycles, that is, four tests in total, to select individuals with symptoms (FIG. 3). During treatment, the symptomatic behavior was investigated with R/I tests at two diestrus and one estrus phase.

Symptoms and video film analysis of symptoms: The behavior of the resident rat was quantified using the Soldis SCORE software version 3.4 (www.soldis.se/company_news.htm, Uppsala Sweden) across five minutes from start of the intrusion (of eight minutes in total). The operator was blinded to the cycle phase and the treatment of each rat and the behaviors of the intruding rat were not analyzed. The analyzed parameters were typical symptoms seen during increased excitability due to tolerance development in animals, e.g. frequency of signs of stress/pain symptoms, irritability/attack symptoms, aversion symptoms against the intruder during the five minutes of intruder test and duration of normal social behavior. Symptoms were confirmed when a rat showed presence of symptomatic behavior at diestrus in at least one of the two tested estrus cycles. The reliability of the scoring of symptom frequency was analyzed by statistically testing the scoring results by two independent operators blinded from each other. The frequencies showed a Pearson's correlation coefficient of r=0.90 and r=0.87 and a Chronbach's alpha of 0.65 and 0.93.

3beta-hydroxy-5alpha-pregnan-20-one treatment: The rats that displayed symptoms were selected for the treatment phase of the study. Treatment was started at estrus, that is one day before the onset of symptoms, and was comprised of daily s.c. injections of 1.5 mg/kg 3beta-hydroxy-5alpha-pregnan-20-one until the second diestrus had passed. In the following two estrus cycles the rats were tested in the R/I model (as above) once at each diestrus and once at estrus (FIG. 3). The daily injections were given in the morning and R/I testing took place 2 hours afterwards. Treatment was continued until sufficient testing was completed.

Statistics: Statistical analyses were performed using SPSS statistics software version 22.0.0.0. One-way analysis of variance (ANOVA) with repeated measures was used to investigate the mean level of symptomatic behaviors across the estrus cycle phases. The test for within-subject's effect was first, the sphericity assumed test, and second (when Mauchly's test of sphericity was significant), the Greenhouse-Geisser test. P values below 0.05 were considered statistically significant and the least significant difference test was used as ad hoc analysis when the overall ANOVA showed significance.

Among the individuals with symptoms, the estrus and diestrus phases of the two cycles prior to treatment were analyzed to establish base-line behaviors. Then, the estrus and diestrus phases tested during treatment were compared to mean values of the pretreatment estrus and diestrus phases, respectively.

### Results

### Base-Line Behavior:

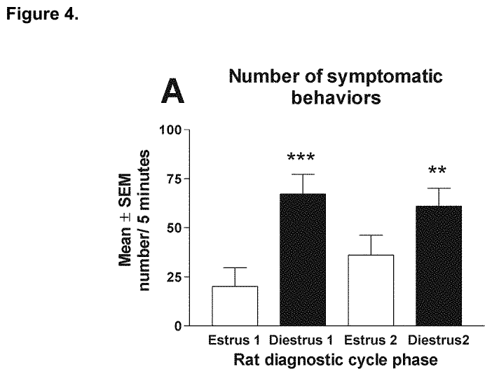

Fourteen (42%) of the 33 tested rats showed signs of symptomatic behavior in at least one of the two tested baseline cycles. In this group (n=14), the symptom frequency was significantly higher at diestrus than at estrus. Analysis of phase-specific mean values of the two cycles combined revealed that symptomatic frequency was significantly higher at diestrus compared to that at estrus (65.2±7.2±SEM vs 26.0±6.7; F(3, 36)=8.131; p<0.001; FIG. 4). No differences were found between the two estrus phases or the two diestrus phases, respectively.

Nine (27%) of the 33 rats showed symptoms in both of the tested cycles. In this group, behavior was more frequent at diestrus than at estrus (F(3,24)=14.086; p<0.001) in both cycles (1^{st} cycle p=0.003, and 2^{nd} cycle p=0.003, respectively).

### Estrus Cycle and Symptomatic Behavior During 3Beta-Hydroxy-5Alpha-Pregnan-20-One Treatment:

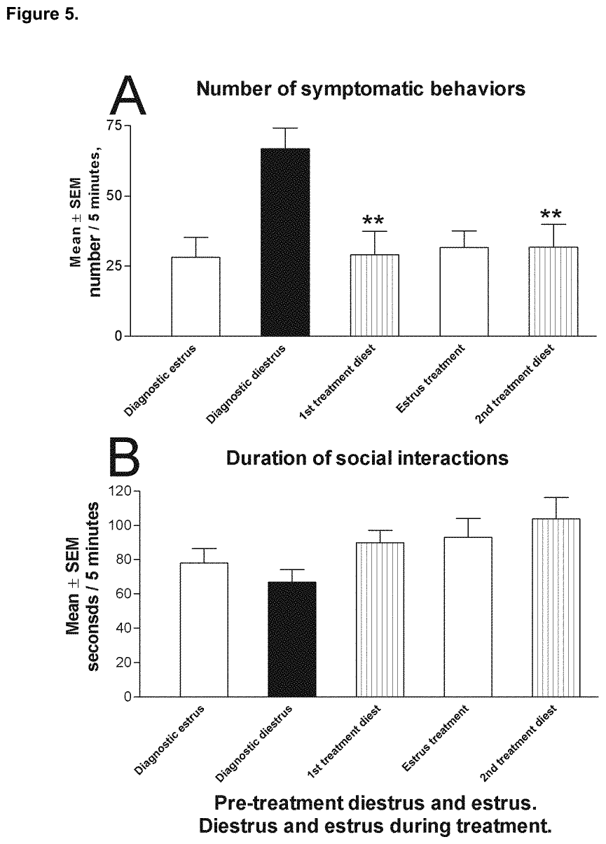

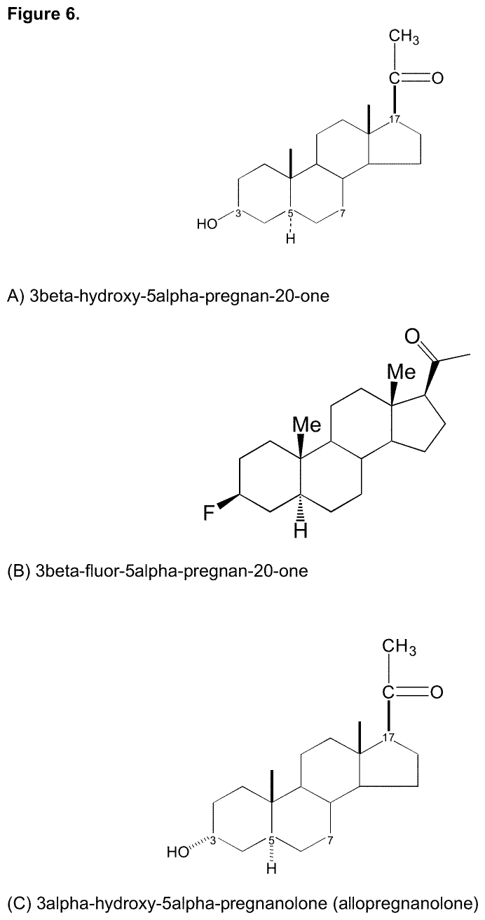

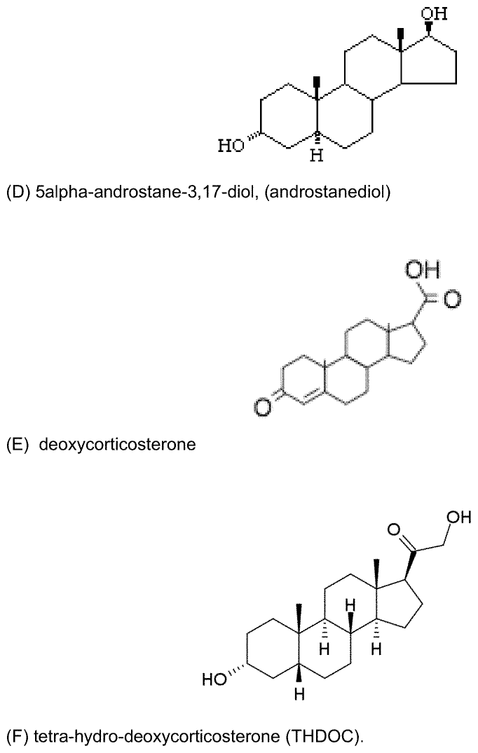

The 14 rats with symptoms were selected for the treatment phase of the study. The estrus cycle duration or vaginal smear appearance did not change during the 3beta-hydroxy-5alpha-pregnan-20-one treatment (data not shown). The display of symptoms at diestrus during treatment with 3beta-hydroxy-5alpha-pregnan-20-one were compared to the mean value of each individual's tolerance symptoms during diestrus at baseline. When the rats were treated with 3beta-hydroxy-5alpha-pregnan-20-one, the symptoms were significantly decreased at diestrus during treatment in comparison to mean diestrus without treatment (F(2,26)=7.127; p=0.003; FIG. 5). The decrease in symptoms at diestrus was observed in both tested cycles, which was confirmed by the ad hoc analysis (1^{st} cycle p=0.005, and 2^{nd} cycle p=0.003). No differences in symptom frequency were found between estrus during treatment and estrus at baseline (FIG. 5A). More importantly, no differences in symptomatic behavior frequency were found between the two diestrus phases during treatment and either estrus during treatment or estrus at baseline. The level of normal social interaction did not differ between the tested estrus cycle phases (FIG. 5B). To calculate the relative effect of the 3beta-hydroxy-5alpha-pregnan-20-one treatment on t symptoms at diestrus the relative effect in symptomatic behavior at diestrus was calculated. When baseline diestrus symptoms was set to 100%, and baseline estrus symptoms was set to 0% the effect of the 3beta-hydroxy-5alpha-pregnan-20-one treatment was −89% and effect size calculated as Cohen's d=1.67.

The group of nine rats that showed symptoms in both of the tested cycles were tested over four test occasions a) individual baseline mean diestrus attack frequency b) diestrus1, c) estrus and d) diestrus2. The symptomatic behaviors were less frequent at both diestrus phases when treated with 3beta-hydroxy-5alpha-pregnan-20-one compared to diestrus at baseline (F(4,32)=7.820; p<0.001, ad hoc: p=0.044 and p=0.005, respectively). The levels of symptoms during treatment were not different compared to either estrus during treatment or estrus at baseline, and there was no difference between the estrus phases. The level of normal social interaction did not differ between the tested estrus cycle phases (data not shown).

### Conclusions

The results show that 3beta-hydroxy-5alpha-pregnan-20-one treatment in female rats significantly reduced diestrus phase induced symptoms frequency, down to levels seen during the non-symptomatic estrus phase, as well as no signs of withdrawal were seen. 3beta-hydroxy-5alpha-pregnan-20-one is thus an effective treatment for reduction of signs of tolerance development and following withdrawal symptoms in adult female rats. The present disclosure thus surprisingly provides a possible treatment of withdrawal disorders as 3beta-hydroxy-5alpha-pregnan-20-one is active as a GABA_{A} steroid modulator antagonist and thus can block the enhancement that the GABA_{A} receptor modulating steroids induce.

### EXAMPLE 4

### Tolerance Development and Withdrawal Disorder can be Hindered by 3Beta-Fluor-5Alpha-Pregnan-20-One (UC2016) Treatment.

The compound by 3beta-fluor-5alpha-pregnan-20-one (UC2016) was analyzed for antagonism of the allopregnanolone-induced loss of righting reflex (LoR) in female Wistar rats.

### Material and Methods

Study design: Allopregnanolone was dissolved 2 mg/ml in 10% 2-hydroxy-beta-cyclodextrin (beta-CD), with the use of ultra sound. 3beta-fluor-5alpha-pregnan-20-one (UC2016) dissolved in MCT (10 mg/ml) was injected s.c. 30 and 60 minutes before i.v. 2 mg/kg allopregnanolone. UC2016, in different concentrations (1-20 mg/kg) were given s.c. 30 or 60 minutes before the acute i.v. allopregnanolone-induced LoR. To study if MCT oil alone interferes with the acute i.v. allopregnanolone-induced LoR, MCT oil was given s.c. 30 or 60 min before the allopregnanolone injection. Doses given were from 1 mg/kg to 20 mg/kg UC2016 s.c.

Animals: Female Wistar rats (n=12) from Taconics, Denmark were used. At arrival rats weighted 250 g. For identification the rats were marked with numbers on their tail with a permanent marker. The animals were housed four in each cage for 6 weeks and thereafter two in each cage at the Umea Centre for Comparative Biology (UCCB) animal facility at Umea University, where also all experiments took place. Housing room temperature was kept at 22-23° C. Lights on and off were monitored 12/12 h light/dark; lights on at 06.00 and off at 18.00. Food and water was available ad libitum. The study protocol was approved by the Regional Ethics Committee of Umeå, Sweden.

Handling: To avoid stress during the experimental session, animals were daily handled the week before testing. Handling included moments used during the test, i.e. holding, tail warming in water, and wrapping in op-blanket.

Solutions: Allopregnanolone (Umecrine AB, batch L00019654), was dissolved in 10% or 30% 2-hydroxypropyl-β-cyclodextrin (HPβ-CD; Sigma, H-107) with the use of ultra sound. MCT oil and Solution of UC2016; 10 mg/ml in MCT oil, were provided by XSpray Microparticles AB (Stockholm).

Allopregnanolone-induced loss of righting reflex (LoR) test: During testing, the rat was weighted, the tail was warmed in water (43° C.) for three minutes, and the rat was wrapped in an op-blanket and hold for injection. The test solution was injected i.v. in one of the tail veins during 30 s (optimal injection rate for allopregnanolone, 4 mg/kg/min, Zhu et al 2001), and the needle then kept in place for additional 30 s. The time point for the end of the actual injection was noted. The rat was then placed in a separate cage and LoR was tested by laying the rat on the right side. Rats with LoR stayed on side, and the time until righting was followed. Rats with no LoR could not be placed on side, as the righting reflex immediate corrected the orientation of the body so that the rat was standing on its paws. The effects of the different injections were followed by the two evaluators for approximately 10 min, and the rat was then taken back to its home cage. LoR is an early event at anesthesia, and this fact is used in the test. Allopregnanolone is a strong anesthetic substance, an agonist at the GABA_{A} receptor, and LoR is in the test induced during the 30 seconds with the injection of the substance. At tests of possible allopregnanolone antagonists, the antagonistic effect is thus observed immediately after the injection, i.e. when the rat is placed in the test cage.

### Results

### Effect of MOT Oil and UC2016 on the Allopregnanolone-Induced Loss of Righting Reflex (LoR)

MOT oil 4 ml/kg s.c. had no effect on allopregnanolone (2 mg/kg i.v.) induced LoR administered either 30 minutes or 60 minutes before allopregnanolone injection. UC2016 was then for this evaluation dissolved 10 mg/ml in MOT. Allopregnanolone 2 mg/kg alone induces LoR in all female Wistar rats.

UC2016, above 1 mg/kg in MCT oil, subcutaneously given 60 minutes before the allopregnanolone injection, inhibits allopregnanolone from inducing LoR (Table 8). Different concentrations of UC2016 and different time intervals between the injections were investigated to characterize the UC2016 inhibitory effect of the allopregnanolone-induced LoR (Table 8). UC2016 at a dosage of 10 mg/kg or higher, 30 min before the allopregnanolone injection, inhibited LoR induction by allopregnanolone, while 5 mg/kg of UC2016, (ratio allopregnanolone:UC2016; 1:2.5) had an inhibitory effect of the allopregnanolone induction of LoR when administered 60 but not 30 minutes before allopregnanolone (Table 8). Thus, UC2016 concentrations below 5 mg/kg, were administrated subcutaneously 60 minutes before the allopregnanolone i.v. The lowest UC2016 concentration tested for LoR antagonism, 1 mg/kg, clearly antagonized the allopregnanolone induction of LoR when given 60 min before allopregnanolone. Data with longer time intervals between the injections are more uncertain (data not shown).

TABLE 8
3beta-fluor-5alpha-pregnan-20-one (UC2016) antagonism of
allopregnanolone-induced loss of righting reflex (LoR).

| UC2016 | Animals (%) not showing LoR after |
|---|---|
| (mg/kg) | allopregnanolone injection |
| 20 | 100% |
| 10 | 100% |
| 2 | 80% |
| 1 | 100% |

### Conclusions

Subcutaneously administered 3beta-fluor-5alpha-pregnan-20-one (UC2016), down to a dosage of 1 mg/kg in MCT oil, inhibited the 2 mg/kg allopregnanolone-induced loss of righting reflex (LoR). In conclusion, results show that 3beta-fluor-5alpha-pregnan-20-one (UC2016) is a potent GAMSA in the inhibition of the allopregnanolone-induced LoR.

### EXAMPLE 5

Study Title: A Pilot Phase II, Randomised, Double-Blind, Placebo-Controlled, Parallel-Group Study Investigating the Efficacy and Safety of Repeated Injections of Sepranolone (UC1010) in Patients with Menstrual Migraine

Study rationale: Migraine is up to three times more common in women than in men, and 50% of women report an association between migraine attacks and menstrual periods at the withdrawal period after long exposure to endogenous allopregnanolone via its effects on the GABA_{A} receptor. Menstrual migraine is therefore considered to be a withdrawal disorder (MacGregor, Hackshaw, 2014). Sepranolone (UC1010) is an endogenous progesterone metabolite isoallopregnanolone (3beta-hydroxy-5alpha-pregnan-20-one), and is a GABA_{A} receptor modulating steroid antagonist (GAMSA). As such, Sepranolone (UC1010) specifically inhibits the action of positive GABA_{A} receptor modulating steroids and thus the tolerance development and withdrawal symptoms in animal studies. The International Headache Society separates the concept of Menstrual Migraine (MM) into menstrually related migraine and pure menstrual migraine. For the purpose of this study the population is women with migraine attacks in at least 2 out of 3 menstrual cycles, with migraine attacks commencing between menses days −2 to +3, defining first day of full menstrual flow as Day 1 (MacGregor 1996, 2006). The women may also have more than occasional-migraine attacks outside of the menstrual period. The prevalence of MM based on the definition in the present study synopsis, should be slightly greater than 8%. UC1010 in the intended doses has been shown safe in clinical pharmacology studies and in a Ph2a study in PreMenstrual Dysphoric Disorder (PMDD). In this study both investigators and patients will be masked for active or placebo treatment in a truly double blind fashion.

### Objectives, Endpoints and Assessments:

Primary objective: Efficacy in reduction of menstrual migraine, Primary Endpoint: Difference between UC1010 and placebo in change from baseline of number of migraine days between Day −2 to +7 during three consecutive menstrual cycles. Assessments: The primary variable in the study will be collected by a daily patient ediary. The diary should collect the following information for each migraine attack as it occurred: date and time of onset of migraine symptoms, peak severity (mild, moderate or severe), duration of attack (to nearest day), associated symptoms (nausea, vomiting, photophobia, phonophobia), aura if present, rescue symptomatic migraine medication (name, dose, time taken). The diary should also include questions on other headaches.

Secondary Objectives: Investigate the treatment efficacy of UC1010 as assessed by amount of symptomatic treatment needed. Investigate difference between UC1010 and placebo in change of number of moderate days compared to severe intensity days. Investigate the responder rate of UC1010 on menstrual migraine. Investigate the effect of UC1010 on the menstrual migraine pain intensity. To evaluate patient satisfaction.

### Study Methodology:

The study is an interventional, multi-center, randomized, double-blind, parallel-group, placebo-controlled, dose-finding study designed to demonstrate proof-of-concept (PoC), that is, that UM1010 can prevent frequency and severity of menstrual migraine.
75 patients (according to power calculation) with confirmed menstrual migraine will be randomly allocated via a centralized blinded randomization system to one of three treatment groups of either 10 mg or 16 mg of UM1010 or placebo with a ratio of 1:1:1.
The target population is women between 18-45 years, suffering from menstrual migraine as defined by recurrent migraine attacks occurring in the withdrawal period during immediate premenstrual and menstrual phase.
The study will start with a baseline period of 3 menstrual cycles, with clinical confirmation of the diagnosis for each individual woman. Patients will not be allowed to be on any other prophylactic treatment for migraine but will be allowed to take rescue medication. The rescue medication will be the patient's standard of care symptomatic treatment on an as-needed basis.
Study treatment is self-administered SC injections every second day, (48 hours), with the first injection starting 14 days prior to the expected onset of next menstruation and ends on the day of full menstrual flow.

The starting day of study treatment with UC1010 will be at the estimated onset of tolerance development and production of allopregnanolone. The treatment starting day is defined by the calendar method and estimated to the first day of the luteal phase. To ensure that the cycle was an ovulatory one, plasma progesterone levels will be measured at a visit on Day −7, when also blood samples for measurement of plasma isoallopregnanolone will be drawn.

Main Inclusion Criteria, The woman must:

give her informed consent to study participation, be aged ≤18 and ≤45 years, have a regular menstrual cycle, use double barrier contraception, non-hormonal IUD, be abstinent or subject or partner has been surgically sterilized, have menstrual migraine as defined in ICHD-3 (2013) in at least 12 mo, for randomisation, have documented menstrual migraine during two of three menstrual cycles at baseline, have maximum ten (10) migraine or other headache days per month.

Main Exclusion Criteria, The woman must not have:

participated in a clinical study within 5 study drug half-lifes, prior to the first study visit, have evidence and/or history of any neurological disease, cancer, chronic infection, allergy or conditions potentially interfering with study assessments, a BMI >35 kg/m². Have a less than 80% compliance with eDiary reporting during the three-cycle baseline period.

The woman must not be: suffering any other medically significant and unstable disease, be or planning pregnancy or lactating, have any steroid hormonal treatment (including hormonal IUD or other local hormonal treatment) during 3 months before the study visit. For injectable hormonal contraceptives, a 6-month washout is required, been diagnosed with a psychiatric disorder according to DSM 5® criteria, including drug abuse or dependency. The woman must not be taking the following concomitant medications (the list is not comprehensive), Prophylactic treatment against migraine, e.g. beta-blocker, anti-epileptics, immunotherapy CGRP-antagonist, triptans for prophylactic use (triptans allowed for symptomatic use), over-the-counter or prescription drugs for PMS symptoms, including but not limited to, bioidentical hormones, St John's wort, Evening primrose oil, Agnus castor.

Sample size calculations: Migraine data from MacGregor (2004) was analysed to estimate the number of women needed in the study. The primary endpoint is the change from baseline to treatment in the mean number of migraine days per cycle in the window of day −2 to +7 (9 days). The observed standard deviation of the change from baseline, i.e. (mean of 3 post treatment cycles−mean of three baseline cycles)—difference was 1.08 days. Assuming an effect size of 0.63 (the number of migraine days per cycle in the active group is 0.68 days less than in the placebo group (the observed mean number of migraine days per cycle at baseline in MacGregor (2004) is 1.7 days), the common standard deviation is 1.08), 24 patients evaluable for efficacy per treatment group (72 patients in total) are needed to achieve a statistical power of 80% using a one-sided t-test at significance level 5% (type I error rate 5%) to test the primary hypothesis (Null hypothesis: no difference between the pooled active group and the placebo group versus the alternative hypothesis: the pooled active group reduces the number of migraine days more than the control group). Assuming a drop-out rate of 20%, recruiting a total sample size of 30 per group into the three treatment groups with a 1:1:1 ratio for the two active dose groups and placebo is expecting to give 24 patients per group evaluable for efficacy.

The present inventors expect that, the study will show that the number of days with migraine during the Allopregnanolone withdrawal period just prior to the menstruation will be lower with the active treatment compared to placebo treatment when the treatment is initiated at the time point of onset of tolerance development (which in the case of menstrual migraine is approximately 14 days prior to the onset of the menstrual bleeding). It is expected that the amount of supplementary symptomatic treatment needed is lower on active treatment compared to placebo and that menstrual migraine pain intensity is lower on active treatment than on placebo. It is expected that the number of patients who experience moderate and severe migraine is lower in the group on active treatment compared to the placebo group. Expected is also an increase in patient satisfaction when on active treatment compared to placebo.

### REFERENCES

Akerman S, Romero-Reyes M, Holland P R. Current and novel insights into the neurophysiology of migraine and its implications for therapeutics. Pharmacol Ther. 2017 April; 172:151-170.
Andreou A P, Shields K G, Goadsby P J. GABA and valproate modulate trigeminovascular nociceptive transmission in the thalamus. Neurobiol Dis. 2010 February; 37(2):314-23. doi: 10.1016/j.nbd
Bäckström T, Epileptic seizures in women in relation to variations of plasma estrogen and progesterone during the menstrual cycle. Acta Neurol Scand 54 (1976) 321-347.
Barnes E M. Use-Dependent Regulation of GABA_{A} Receptors. International Review of Neurobiology 39; 1996:53-76, 1996.
Birzniece V, Turkmen S, Lindblad C, Zhu D, Johansson I M, Bäckström T, Wahlstrom G. GABA-A receptor mRNA changes in acute allopregnanolone tolerance. European Journal of Pharmacology 2006 Mar. 27; 535(1-3):125-34.
Brust J C M (2004). Neurological aspects of substance abuse. Elsevier Philadelphia 2004 pp 1-455; ISBN-O-7506-7313-3.
Cutrer F M, Limmroth V, Ayata G, Moskowitz M A, Attenuation by valproate of c-fos immunoreactivity in trigeminal nucleus caudalis induced by intracisternal capsaicin, Br. J. Pharmacol. 116 (1995) 3199-3204.
Droogleever Fortuyn H A, van Broekhoven F, Verkes R J, Bäckström T, Zitman F G, Span P N. Effects of PhD examination stress on allopregnanolone and cortisol plasma levels and peripheral benzodiazepine receptors. Psychneuroendocrinology 2004; 29:1341-4
Drower E J, Hammond D L, GABAergic modulation of nociceptive threshold: effects of THIP and bicuculline microinjected in the ventral medulla of the rat, Brain Res. 450 (1988) 316-324.
Headache Classification Committee of the International Headache Society (IHS). The International Classification of Headache Disorders, 3rd edition. Cephalalgia. 2018 January; 38(1):1-211. doi: 10.1177/0333102417738202.
Hubscher, C. H., Brooks, D. L., Johnson, J. R., 2005. A quantitative method for assessing stages of the rat estrous cycle. Biotechnic & histochemistry: official publication of the Biological Stain Commission 80, 79-87.
Ichikawa S, Sawada T, Nakamura Y, Morioka H. Ovarian secretion of pregnane compounds during the estrus cycle and pregnant rats. Endocrinology 94; 1974: 1615-1620.
Laidlaw, J. (1956): Catamenial Epilepsy, Lancet 271, 1235-1237. Bäckström T, Epileptic seizures in women in relation to variations of plasma estrogen and progesterone during the menstrual cycle. Acta Neurol Scand 54 (1976) 321-347
MacGregor E A. Menstrual migraine: therapeutic approaches. Ther Adv Neurol Disord (2009) 2(5) 327-336.
MacGregor E A. “Menstrual” migraine: towards a definition. Cephalalgia 1996; 16:11-21. Cephalalgia. 1996 February; 16(1):11-21.
MacGregor E A, Hackshaw A, Prevalence of migraine on each day of the natural menstrual cycle, Neurology. 2014; 63:351-353.
MacGregor E A, Frith A, Ellis J, Aspinall L, Hackshaw A. Incidence of migraine relative to menstrual cycle phases of rising and falling estrogen. Neurology. 2006; 67(12):2154-8.
O'Keane V, Lightman S, Patrick K, Marsh M, Papadopoulos A S, Pawlby S, Seneviratne G, Taylor A, Moore R. Changes in the maternal hypothalamic-pituitary-adrenal axis during the early puerperium may be related to the postpartum ‘blues’. J Neuroendocrinol. 2011 November; 23(11):1149-55.
Olsen R W. GABAA receptor: Positive and negative allosteric modulators. Neuropharmacology. 2018 Jan. 30. pii: S0028-3908(18)30036-4. doi: 10.1016/j.neuropharm.2018.01.036.
Prince R J, Simmons M A. Differential antagonism by epipregnanolone of alphaxalone and pregnanolone potentiation of [3H]flunitrazepam binding suggests more than one class of binding site for steroids at GABAA receptorsNeuropharmacology, 1993; 32; 59-63.
Reddy D S, Gould J, Gangisetty O. A mouse kindling model of perimenstrual catamenial epilepsy. J Pharmacol Exp Ther. 2012 June; 341(3):784-93.
Reddy D S, Rogawski M A. Neurosteroid replacement therapy for catamenial epilepsy. Neurotherapeutics. 2009 April; 6(2):392-401.
Reddy D S. The role of neurosteroids in the pathophysiology and treatment of catamenial epilepsy. Epilepsy Res. 2009 July; 85(1):1-30.
Schneider, T., Popik, P., 2007. Attenuation of estrous cycle-dependent marble burying in female rats by acute treatment with progesterone and antidepressants. Psychoneuroendocrinology 32, 651-659.
Slawecki C J, Purdy R H, Li T K, Walpole T, Ehlers C L. Effects of allopregnanolone on the EEG of alcohol-preferring and alcohol-nonpreferring rats. Alcohol Clin Exp Res. 2000 September; 24(9):1369-75.
Smith S S, Gong Q H, Hsu F C, Markowitz R S, ffrench-Mullen J M H, Li X. GABAA receptor alpha4 subunit suppression prevents withdrawal properties of an endogenous steroid. NATURE 392; 1998: 926-930.
Storer R J, Akerman S, Shields K G, Goadsby P J. GABAA receptor modulation of trigeminovascular nociceptive neurotransmission by midazolam is antagonized by flumazenil. Brain Res. 2004 Jul. 9; 1013(2):188-93.
Storer, R. J. Akerman S, Goadsby P J. GABA receptors modulate trigeminovascular nociceptive neurotransmission in the trigeminocervical complex, Br. J. Pharmacol. 134 (2001) 896-904.
Turkmen S, Backstrom T, Wahlstrom G, Andreen L, Johansson I M. Tolerance to allopregnanolone with focus on the GABA-A receptor. Br J Pharmacol. 2011 January; 162(2):311-27.
Vanini G, Watson C J, Lydic R and Baghdoyan H A (2008). Gamma-aminobutyric acid-mediated neurotransmission in the pontine reticular formation modulates hypnosis, immobility, and breathing during isoflurane anesthesia. Anesthesiology 109: 978-988, 2008.
Vetvik K G, Benth J B, MacGregor E A, Lundqvist C, Russell M B. Menstrual versus non-menstrual attacks of migraine without aura in women with and without menstrual migraine. Cephalalgia. 2015 December; 35(14):1261-8.
Wang M, He Y, Eisenman L N, Fields C, Zeng C M, Mathews J, Benz A, Fu T, Zorumski E, Steinbach J H, Covey D F, Zorumski C F, Mennerick S. 3beta-Hydroxypregnane Steroids Are Pregnenolone Sulfate-Like GABAA Receptor Antagonists. The Journal of Neuroscience, 2002; 22(9):3366-3375.
Wang M. D., Bäckström T. Landgren S. The inhibitory effects of allopregnanolone and pregnanolone on the population spike, evoked in the rat hippocampal CA1 stratum pyramidale in vitro, can be blocked selectively by epiallopregnanolone. Acta Physiol Scand. 2000; 169:333-341.
Welch K M. Brain hyperexcitability: the basis for antiepileptic drugs in migraine prevention. Headache. 2005 April; 45 Suppl 1:S25-32.
Wisden W, Laurie D J, Monyer H, Seeburg P H. The distribution of 13 GABAA receptor subunit mRNAs in the rat brain. I. Telencephalon, diencephalon, mesencephalon. J Neurosci. 1992 March; 12(3):1040-62.
Wolf B, Griffiths R R. Physical dependence on benzodiazepines: differences within the class. Drug Alcohol Depend. Drug Alcohol Depend. 1991 Dec. 31; 29(2):153-6 Zhu D., Birzniece V., Bäckström T. and Wahlstrom G. (2004) Dynamic aspects of acute tolerance to allopregnanolone evaluated using anaesthesia threshold in male rats. British Journal of Anaesthesia 93, 560-567.

### Items of the Invention

1. GABA_{A} receptor modulating steroid antagonist for use in treatment of a substance withdrawal disorder.
2. GABA_{A} receptor modulating steroid antagonist for use according to item 1, wherein said substance withdrawal disorder is associated with and/or attributed to and/or caused by withdrawal of a positive GABA_{A} receptor modulating steroid.
3. GABA_{A} receptor modulating steroid antagonist for use according to item 1 or 2, wherein said positive GABA_{A} receptor modulating steroid is selected from the group consisting of allopregnanolone, androstanediol, deoxycorticosterone and any combination thereof; such as the group consisting of allopregnanolone, deoxycorticosterone and any combination thereof.
4. GABA_{A} receptor modulating steroid antagonist for use according to item 3, wherein said deoxycorticosterone is tetra-hydro-deoxycorticosterone (THDOC).
5. GABA_{A} receptor modulating steroid antagonist for use according to any one of items 1-4, wherein said treatment is preventive treatment.
6. The GABA_{A} receptor modulating steroid antagonist for use, according to any one of items 1-5, wherein said disorder is headache associated with and/or attributed to substance withdrawal.
7. The GABA_{A} receptor modulating steroid antagonist for use, according to item 6, wherein said disorder is headache associated with and/or attributed to allopregnanolone withdrawal.
8. The GABA_{A} receptor modulating steroid antagonist for use, according to any one of items 6-7, wherein said disorder is headache associated with and/or attributed to androstanediol withdrawal.
9. The GABA_{A} receptor modulating steroid antagonist for use, according to any one of items 6-8, wherein said disorder is headache associated with and/or attributed to deoxycorticosterone, such as tetra-hydro-deoxycorticosterone (THDOC), withdrawal.
10. The GABA_{A} receptor modulating steroid antagonist for use, according to any one of items 1-5, wherein said disorder is migraine attributed to substance withdrawal.
11. The GABA_{A} receptor modulating steroid antagonist for use, according to item 10, wherein said disorder is migraine associated with and/or attributed to allopregnanolone withdrawal.
12. The GABA_{A} receptor modulating steroid antagonist for use, according to any one of items 10-11, wherein said disorder is migraine associated with and/or attributed to androstanediol withdrawal.
13. The GABA_{A} receptor modulating steroid antagonist for use, according to any one of items 11-12, wherein said disorder is migraine associated with and/or attributed to deoxycorticosterone, such as tetra-hydro-deoxycorticosterone (THDOC), withdrawal.
14. The GABA_{A} receptor modulating steroid antagonist for use, according to any one of items 1-5, wherein said disorder is a seizure disorder associated with and/or attributed to substance withdrawal.
15. The GABA_{A} receptor modulating steroid antagonist for use, according to item 14, wherein said disorder is a seizure disorder associated with and/or attributed to allopregnanolone withdrawal.
16. The GABA_{A} receptor modulating steroid antagonist for use, according to any one of items 14-15, wherein said disorder is a seizure disorder associated with and/or attributed to androstanediol withdrawal.
17. The GABA_{A} receptor modulating steroid antagonist for use, according to any one of items 14-16, wherein said disorder is a seizure disorder associated with and/or attributed to deoxycorticosterone, such as tetra-hydro-deoxycorticosterone (THDOC), withdrawal.
18. The GABA_{A} receptor modulating steroid antagonist for use, according to any one of items 1-5, wherein said disorder is an anxiety disorder associated with and/or attributed to substance withdrawal.
19. The GABA_{A} receptor modulating steroid antagonist for use, according to item 18, wherein said disorder is an anxiety disorder associated with and/or attributed to allopregnanolone withdrawal.
20. The GABA_{A} receptor modulating steroid antagonist for use, according to any one of items 18-19, wherein said disorder is an anxiety disorder associated with and/or attributed to androstanediol withdrawal.
21. The GABA_{A} receptor modulating steroid antagonist for use, according to any one of items 18-20, wherein said disorder is an anxiety disorder associated with and/or attributed to deoxycorticosterone, such as tetra-hydro-deoxycorticosterone (THDOC), withdrawal.
22. The GABA_{A} receptor modulating steroid antagonist for use, according to any one of items 1-5, wherein said disorder is a panic disorder associated with and/or attributed to substance withdrawal.
23. The GABA_{A} receptor modulating steroid antagonist for use, according to item 22, wherein said disorder is a panic disorder associated with and/or attributed to allopregnanolone withdrawal.
24. The GABA_{A} receptor modulating steroid antagonist for use, according to any one of items 22-23, wherein said disorder is a panic disorder associated with and/or attributed to androstanediol withdrawal.
25. The GABA_{A} receptor modulating steroid antagonist for use, according to any one of items 22-24, wherein said disorder is a panic disorder associated with and/or attributed to deoxycorticosterone, such as tetra-hydro-deoxycorticosterone (THDOC), withdrawal.
26. GABA_{A} receptor modulating steroid antagonist for use, according to any one of items 1-5, wherein said disorder is a depression associated with and/or attributed to substance withdrawal.
27. GABA_{A} receptor modulating steroid antagonist for use, according to item 26, wherein said disorder is a depression associated with and/or attributed to allopregnanolone withdrawal.
28. GABA_{A} receptor modulating steroid antagonist for use, according to any one of items 26-27, wherein said disorder is a depression associated with and/or attributed to androstanediol withdrawal.
29. GABA_{A} receptor modulating steroid antagonist for use, according to any one of items 26-28, wherein said disorder is a depression associated with and/or attributed to deoxycorticosterone withdrawal, such as tetra-hydro-deoxycorticosterone (THDOC) withdrawal.
30. The GABA_{A} receptor modulating steroid antagonist for use, according to any one of items 1-29, said GABA_{A} receptor modulating steroid antagonist being 3beta-hydroxy-5alpha-pregnan-20-one, or a pharmaceutically acceptable salt thereof.
31. The GABA_{A} receptor modulating steroid antagonist for use, according to any one of items 1-29, said GABA_{A} receptor modulating steroid antagonist being 3beta-fluor-5alpha-pregnan-20-one, or a pharmaceutically acceptable salt thereof.
32. The GABA_{A} receptor modulating steroid antagonist for use, according to any one of items 1-31, provided in combination with one or more compounds selected from: acetaminophen, ibuprofen, acetylsalicylic acid, naproxen sodium, diclofenac potassium, almotriptan, eletriptan, frovatriptan, naratriptan, rizatriptan, sumatriptan, and zolmitriptan, dihydroergotamine, non-opioid acetaminophen, caffeine, metoclopramide, domperidone, prochlorperazine, etoprolol, propranolol, onabotulinumtoxin A, amitriptyline, venlafaxine, fluoxetine, topiramate, lamotrigine, gabapentin carbamazepine, Erenumab and Fremanezumab-vfrm.
33. GABA_{A} receptor modulating steroid antagonist for use, according to any one of items 1-32, wherein said treatment takes place before the manifestation of said substance withdrawal disorder.
34. GABA_{A} receptor modulating steroid antagonist for use, according to any one of items 1-33, wherein said treatment is initiated no later than at a time point of onset of tolerance development to the substance.
35. GABA_{A} receptor modulating steroid antagonist for use, according to any one of items 1-34, wherein said treatment is initiated prior to the development of tolerance to the substance.
36. GABA_{A} receptor modulating steroid antagonist for use, according to any one of items 1-35, wherein said treatment is initiated at a time point prior to the manifestation, such as the expected manifestation, of said withdrawal disorder in a patient, such as a patient who is not undergoing treatment with GABA_{A} receptor modulating steroid antagonist.
37. GABA_{A} receptor modulating steroid antagonist for use, according to any one of items 1-36, wherein said treatment is initiated at least about 1 day before the expected manifestation of the substance withdrawal disorder.
38. GABA_{A} receptor modulating steroid antagonist for use, according to any one of items 1-37, wherein said treatment is initiated at a time point at least about 2 to about 3 days before the manifestation, such as the expected manifestation, of the substance withdrawal disorder.
39. GABA_{A} receptor modulating steroid antagonist for use, according to any one of items 1-38, wherein said treatment is initiated at a time point of from about 2 to about 21 days, such as from about 2 to about 20 days, such as about 2 to about 19 days, such as about 2 to about 18 days, such as about 2 to about 17 days, such as about 2 to about 16 days, such as about 2 to about 15 days, such as about 2 to about 14 days, such as about 2 to about 13 days, such as about 2 to about 12 days, such as about 2 to about 11 days, such as about 2 to about 10 days, such as about 2 to about 9 days, such as about 2 to about 8 days, such as about 2 to about 7 days, such as about 2 to about 6 days, such as about 2 to about 5 days, such as about 2 to about 4 days, such as about 2 to about 3 days prior to the manifestation, such as the expected manifestation, of said substance withdrawal disorder.
40. GABA_{A} receptor modulating steroid antagonist for use, according to any one of items 1-38, wherein said time point is at least about 1 week, such as at least about 2 weeks, such as at least about 3 weeks, such as at least about 4 weeks, such as at least about 5 weeks, such as at least about 6 weeks, such as at least about 7 weeks, such as at least about 8 weeks.
41. GABA_{A} receptor modulating steroid antagonist for use, according to any one of items 1-38, wherein said time point is at least about 1 month, such as at least about 2 months, such as at least about 3 months, such as at least about 4 months.
42. GABA_{A} receptor modulating steroid antagonist for use, according to any one of items 34-41, wherein said time point encompasses the time range of +/−25%, such as the time range +/−20%, such as the time range +/−15%, such as the time range +/−10%.
43. GABA_{A} receptor modulating steroid antagonist for use, according to any one of items 1-39 and 42, wherein said treatment is initiated at a time point of from about 10 to about 16 days, such as from about 10 to about 14 days, such as from about 11 to about 13 days, such as from about 12 days prior to the expected manifestation of the symptom(s) of the substance withdrawal disorder.
44. GABA_{A} receptor modulating steroid antagonist for use, according to item 43, wherein said substance withdrawal disorder is headache, migraine, seizure(s), depression, anxiety and/or panic, associated with and/or attributed to allopregnanolone withdrawal, such as headache and/or migraine associated with and/or attributed to allopregnanolone withdrawal.
45. GABA_{A} receptor modulating steroid antagonist for use, according to item 43 or 44, wherein said substance withdrawal disorder is a menstrual cycle related substance withdrawal disorder.
46. GABA_{A} receptor modulating steroid antagonist for use, according to any one of items 1-38, 40 and 42, wherein treatment is initiated at a time point of from about 3 weeks +/−1 week, prior to the expected manifestation of the symptoms of the substance withdrawal disorder.
47. GABA_{A} receptor modulating steroid antagonist for use, according to item 46, wherein said substance withdrawal disorder is headache, migraine, seizure(s), depression, anxiety and/or panic, associated with and/or attributed to allopregnanolone withdrawal, such as headache and/or migraine associated with and/or attributed to allopregnanolone withdrawal.
48. GABA_{A} receptor modulating steroid antagonist for use, according to item 46 or 47, wherein said substance withdrawal disorder is a post-partum related substance withdrawal disorder.
49. GABA_{A} receptor modulating steroid antagonist for use, according to any one of items 1-37 and 42, wherein treatment is initiated at a time point of from about 1 to about 3 days, such as about 1, 2 or 3 days, prior to the expected manifestation of the symptoms of the substance withdrawal disorder.
50. GABA_{A} receptor modulating steroid antagonist for use, according to item 49, wherein said substance withdrawal disorder is headache, migraine, seizure(s), depression, anxiety and/or panic, associated with and/or attributed to allopregnanolone and/or THDOC withdrawal, such as headache and/or migraine associated with and/or attributed to allopregnanolone and/or THDOC withdrawal.
51. GABA_{A} receptor modulating steroid antagonist for use, according to item 49 or 50, wherein said substance withdrawal disorder is a stress related substance withdrawal disorder.
52. GABA_{A} receptor modulating steroid antagonist for use, according to any one of items 1-38, 41 and 42, wherein treatment is initiated at a time point of from about 1 to about 6 months, such as about 6, 5, 4, 3, 2 or 1 month days, prior to the expected manifestation of the symptoms of the substance withdrawal disorder.
53. GABA_{A} receptor modulating steroid antagonist for use, according to item 52, wherein said substance withdrawal disorder is headache, migraine, seizure(s), depression, anxiety and/or panic, associated with and/or attributed to androstanediol withdrawal, such as headache and/or migraine associated with and/or attributed to androstanediol withdrawal.
54. GABA_{A} receptor modulating steroid antagonist for use, according to item 52 or 53, wherein said substance withdrawal disorder is a season related substance withdrawal disorder.
55. GABA_{A} receptor modulating steroid antagonist for use, according to any one of the preceding items, wherein said GABA_{A} receptor modulating steroid antagonist is provided in the form of a pharmaceutical composition.
56. GABA_{A} receptor modulating steroid antagonist for use, according to any one of the preceding items, wherein said GABA_{A} receptor modulating steroid antagonist is provided together with a pharmaceutically acceptable excipient, adjuvant or carrier to form a pharmaceutical composition.
57. A method of treating, preventing and/or alleviating a substance withdrawal disorder, comprising administering a pharmaceutically effective amount of a GABA_{A} receptor modulating steroid antagonist, to a patient in need thereof.
58. The method according to item 57, wherein said substance withdrawal disorder is associated with and/or attributed to and/or caused by withdrawal of a positive GABA_{A} receptor modulating steroid.
59. The method according to item 57 or 58, wherein said positive GABA_{A} receptor modulating steroid is selected from the group consisting of allopregnanolone, androstanediol and deoxycorticosterone group consisting of allopregnanolone, androstanediol, deoxycorticosterone and any combination thereof; such as the group consisting of allopregnanolone, deoxycorticosterone and any combination thereof.
60. The method according to item 59 wherein said deoxycorticosterone is tetra-hydro-deoxycorticosterone (THDOC).
61. The method according to any one of items 57-60 wherein said method is a method of preventing a substance withdrawal disorder.
62. The method according to any one of items 57-61, wherein said disorder is headache associated with and/or attributed to substance withdrawal.
63. The method according to item 62, wherein said disorder is headache associated with and/or attributed to allopregnanolone withdrawal.
64. The method according to any one of items 62-63, wherein said disorder is headache associated with and/or attributed to androstanediol withdrawal.
65. The method according to any one of items 62-64, wherein said disorder is headache associated with and/or attributed to deoxycorticosterone, such as tetra-hydro-deoxycorticosterone (THDOC), withdrawal.
66. The method according to any one of items 57-61, wherein said disorder is migraine associated with and/or attributed to substance withdrawal.
67. The method according to item 66, wherein said disorder is migraine associated with and/or attributed to allopregnanolone withdrawal.
68. The method according to any one of items 66-67 wherein said disorder is migraine associated with and/or attributed to androstanediol withdrawal.
69. The method according to any one of items 66-68 wherein said disorder is migraine associated with and/or attributed to deoxycorticosterone, such as tetra-hydro-deoxycorticosterone (THDOC), withdrawal.
70. The method according to any one of items 57-61, wherein said disorder is a seizure disorder associated with and/or attributed to substance withdrawal.
71. The method according to item 70, wherein said disorder is a seizure disorder associated with and/or attributed to allopregnanolone withdrawal.
72. The method according to any one of items 70-71, wherein said disorder is a seizure disorder associated with and/or attributed to androstanediol withdrawal.
73. The method according to any one of items 70-72, wherein said disorder is a seizure disorder associated with and/or attributed to deoxycorticosterone, such as tetra-hydro-deoxycorticosterone (THDOC), withdrawal.
74. The method according to any one of items 57-64, wherein said disorder is an anxiety disorder associated with and/or attributed to substance withdrawal.
75. The method according to item 74, wherein said disorder is an anxiety disorder associated with and/or attributed to allopregnanolone withdrawal.
76. The method according to any one of items 74-75, wherein said disorder is an anxiety disorder associated with and/or attributed to androstanediol withdrawal.
77. The method according to any one of items 74-76, wherein said disorder is an anxiety disorder associated with and/or attributed to deoxycorticosterone, such as tetra-hydro-deoxycorticosterone (THDOC), withdrawal.
78. The method according to any one of items 57-61, wherein said disorder is a panic disorder associated with and/or attributed to substance withdrawal.
79. The method according to item 78, wherein said disorder is a panic disorder associated with and/or attributed to allopregnanolone withdrawal.
80. The method according to any one of items 78-79, wherein said disorder is a panic disorder associated with and/or attributed to androstanediol withdrawal.
81. The method according to any one of items 78-80, wherein said disorder is a panic disorder associated with and/or attributed to deoxycorticosterone, such as tetra-hydro-deoxycorticosterone (THDOC), withdrawal.
82. The method according to any one of items 57-61, wherein said disorder is a depression associated with and/or attributed to substance withdrawal.
83. The method according to item 82, wherein said disorder is a depression associated with and/or attributed to allopregnanolone withdrawal.
84. The method according to any one of items 82-83, wherein said disorder is a depression associated with and/or attributed to androstanediol withdrawal.
85. The method according to any one of items 82-84, wherein said disorder is a depression associated with and/or attributed to deoxycorticosterone withdrawal, such as tetra-hydro-deoxycorticosterone (THDOC) withdrawal.
86. The method according to any one of items 57-85, wherein said GABA_{A} receptor modulating steroid antagonist is 3beta-hydroxy-5alpha-pregnan-20-one, or a pharmaceutically acceptable salt thereof.
87. The method according to any one of items 57-85, wherein said GABA_{A} receptor modulating steroid antagonist is 3beta-fluor-5alpha-pregnan-20-one, or a pharmaceutically acceptable salt thereof.
88. The method according to any one of items 57-87, said compound being provided in combination with one or more compounds selected from: acetaminophen, ibuprofen, acetylsalicylic acid, naproxen sodium, diclofenac potassium, almotriptan, eletriptan, frovatriptan, naratriptan, rizatriptan, sumatriptan, and zolmitriptan, dihydroergotamine, non-opioid acetaminophen, caffeine, metoclopramide, domperidone, prochlorperazine, etoprolol, propranolol, onabotulinumtoxin A, amitriptyline, venlafaxine, fluoxetine, topiramate, lamotrigine, gabapentin, carbamazepine, Erenumab and Fremanezumab-vfrm.
89. The method according to any one of items 57 to 88, wherein said treatment takes place before the manifestation of said substance withdrawal disorder.
90. The method according to any one of items 57 to 89, wherein the treatment is initiated no later than at the time point of onset of tolerance development to the substance.
91. Method according to any one of items 57 to 90, wherein said treatment is initiated prior to the development of tolerance to the substance.
92. Method according to any one of items 57 to 91, wherein said treatment is initiated at a time point prior to the manifestation, such as the expected manifestation, of said withdrawal disorder in a patient who is not undergoing treatment with GABA_{A} receptor modulating steroid antagonist.
93. Method according to any one of items 57 to 92, wherein said treatment is initiated at least about 1 day before the expected manifestation of the substance withdrawal disorder.
94. Method according to any one of items 57 to 93, wherein said treatment is initiated at a time point at least about 2 to about 3 days before the expected manifestation of the substance withdrawal disorder.
95. Method according to any one of items 57 to 94, wherein said treatment is initiated at a time point of from about 2 to about 21 days, such as from about 2 to about 20 days, such as from about 2 to about 19 days, such as from about 2 to about 18 days, such as from about 2 to about 17 days, such as from about 2 to about 16 days, such as from about 2 to about 15 days, such as from about 2 to about 14 days, such as from about 2 to about 13 days, such as from about 2 to about 12 days, such as from about 2 to about 11 days, such as from about 2 to about 10 days, such as from about 2 to about 9 days, such as from about 2 to about 8 days, such as from about 2 to about 7 days, such as from about 2 to about 6 days, such as from about 2 to about 5 days, such as from about 2 to about 4 days, such as from about 2 to about 3 days prior to the manifestation, such as the expected manifestation, of said substance withdrawal disorder.
96. Method according to any one of items 57 to 94, wherein said time point is at least about 1 week, such as at least about 2 weeks, such as at least about 3 weeks, such as at least about 4 weeks, such as at least about 5 weeks, such as at least about 6 weeks, such as at least about 7 weeks, such as at least about 8 weeks.
97. Method according to any one of items 57 to 95, wherein said time point is at least about 1 month, such as at least about 2 months, such as at least about 3 months, such as at least about 4 months.
98. Method according to any one of items 93-97, wherein said time point encompasses the time range of +/−25%, such as the time range +/−20%, such as the time range +/−15%, such as the time range +/−10%.
99. Method according to any one of items 57-95 and 98, wherein said treatment is initiated at a time point of from about 10 to about 16 days, such as from about 10 to about 14 days, such as from about 11 to about 13 days, such as about 12 days prior to the expected manifestation of the substance withdrawal disorder.
100. Method according to item 99, wherein said substance withdrawal disorder is headache, migraine, seizure(s), depression, anxiety and/or panic, associated with and/or attributed to allopregnanolone withdrawal, such as headache and/or migraine associated with and/or attributed to allopregnanolone withdrawal.
101. Method according to item 99 or 100, wherein said substance withdrawal disorder is a menstrual cycle related substance withdrawal disorder.
102. Method according to any one of items 57-97 and 100, wherein treatment is initiated at a time point of about 3 weeks +/−1 week, prior to the expected manifestation of the symptoms of the substance withdrawal disorder.
103. Method according to item 102, wherein said substance withdrawal disorder is headache, migraine, seizure(s), depression, anxiety and/or panic, associated with and/or attributed to allopregnanolone withdrawal, such as headache and/or migraine associated with and/or attributed to allopregnanolone withdrawal.
104. Method according to item 102 or 103, wherein said substance withdrawal disorder is a post-partum related substance withdrawal disorder.
105. Method according to any one of items 57-93 and 100, wherein treatment is initiated at a time point of from about 1 to about 3 days, such as about 1, 2 or 3 days, prior to the expected manifestation of the symptom(s) of the substance withdrawal disorder.
106. Method according to item 105, wherein said substance withdrawal disorder is headache, migraine, seizure(s), depression, anxiety and/or panic, associated with and/or attributed to allopregnanolone and/or THDOC withdrawal, such as headache and/or migraine associated with and/or attributed to allopregnanolone and/or THDOC withdrawal.
107. Method according to item 105 or 106, wherein said substance withdrawal disorder is a stress related substance withdrawal disorder.
108. Method according to any one of items 57-94, 97 and 100, wherein treatment is initiated at a time point of from about 1 to about 6 months, such as about 6, 5, 4, 3, 2 or 1 month, prior to the expected manifestation of the symptoms of the substance withdrawal disorder.
109. Method according to item 108, wherein said substance withdrawal disorder is headache, migraine, seizure(s), depression, anxiety and/or panic, associated with and/or attributed to androstanediol withdrawal, such as headache and/or migraine associated with and/or attributed to androstanediol withdrawal.
110. Method according to item 108 and 109, wherein said substance withdrawal disorder is a season related substance withdrawal disorder.
111. Method according to any one of items 57-110, wherein said GABA_{A} receptor modulating steroid antagonist is provided in the form of a pharmaceutical composition.
112. Method according to any one items 57-111, wherein said GABA_{A} receptor modulating steroid antagonist is provided together with a pharmaceutically acceptable excipient, adjuvant or carrier to form a pharmaceutical composition.
113. Use of a GABA_{A} receptor modulating steroid antagonist in the manufacture of a medicament for the treatment of a substance withdrawal disorder, such as for the preventive treatment of a substance withdrawal disorder.
114. Use of a GABA_{A} receptor modulating steroid antagonist in the manufacture of a medicament according to item 113, wherein said substance withdrawal disorder is associated with and/or caused by withdrawal of a positive GABA_{A} receptor modulating steroid.
115. Use according to item 113 or 114, wherein said positive GABA_{A} receptor modulating steroid is selected from the group consisting of allopregnanolone, androstanediol, deoxycorticosterone and any combination thereof; such as the group consisting of allopregnanolone, deoxycorticosterone and any combination thereof.
116. Use according to item 115, wherein said deoxycorticosterone is tetra-hydro-deoxycorticosterone (THDOC).
117. Use according to any one of items 113-116, wherein said disorder is selected from the group consisting of headache associated with and/or caused by substance withdrawal, migraine associated with and/or caused by substance withdrawal, seizure(s) associated with and/or caused by substance withdrawal, anxiety associated with and/or caused by substance withdrawal, panic associated with and/or caused by substance withdrawal, depression associated with and/or caused by substance withdrawal, and any combination thereof.
118. Use according to any one of items 113-117, wherein said GABA_{A} receptor modulating steroid antagonist is 3beta-hydroxy-5alpha-pregnan-20-one, or a pharmaceutically acceptable salt thereof, and/or 3beta-fluor-5alpha-pregnan-20-one, or a pharmaceutically acceptable salt thereof.
119. Use according to any one of items 113-118, wherein said GABA_{A} receptor modulating steroid antagonist is provided in combination with one or more compounds one or more compounds selected from: acetaminophen, ibuprofen, acetylsalicylic acid, naproxen sodium, diclofenac potassium, almotriptan, eletriptan, frovatriptan, naratriptan, rizatriptan, sumatriptan, and zolmitriptan, dihydroergotamine, non-opioid acetaminophen, caffeine, metoclopramide, domperidone, prochlorperazine, etoprolol, propranolol, onabotulinumtoxin A, amitriptyline, venlafaxine, fluoxetine, topiramate, lamotrigine, gabapentin, carbamazepine, Erenumab and Fremanezumab-vfrm.
120. Use according to any one of items 113-118, wherein said treatment takes place before the manifestation, such as the expected manifestation, of said withdrawals substance disorder.
121. Use according to any one of items 113-120, wherein said GABA_{A} receptor modulating steroid antagonist is provided together with a pharmaceutically acceptable carrier, excipient and/or carrier, i.e. in the form of a pharmaceutical composition.
122. A pharmaceutical composition comprising a GABA_{A} receptor modulating steroid antagonist, for use according to any one of items 1 to 56, together with pharmaceutically acceptable carriers, excipients and/or diluents.
123. The pharmaceutical composition for use, according to item 122, wherein said GABA_{A} receptor modulating steroid antagonist is 3beta-hydroxy-5alpha-pregnan-20-one, or a pharmaceutically acceptable salt thereof.
124. The pharmaceutical composition for use, according to item 122, wherein said GABA_{A} receptor modulating steroid antagonist is 3beta-fluor-5alpha-pregnan-20-one, or a pharmaceutically acceptable salt thereof.

1. A method of treating or preventing a substance withdrawal disorder in a patient, comprising administering to the patient an effective amount of a GABA_{A} receptor modulating steroid antagonist.

2. A method according to claim 1, wherein said substance withdrawal disorder is associated with and/or caused by withdrawal of a positive GABA_{A} receptor modulating steroid.

3. A method according to claim 2, wherein said a positive GABAA receptor modulating steroid is selected from the group consisting of allopregnanolone, androstanediol, and tetrahydrodeoxycorticosterone.

4. A method according to claim 1, wherein said substance withdrawal disorder is selected from the group consisting of headache associated with and/or attributed to substance withdrawal, migraine associated with and/or attributed to substance withdrawal, a seizure disorder associated with and/or attributed to substance withdrawal, an anxiety disorder associated with and/or attributed to substance withdrawal, and a panic disorder associated with and/or attributed to substance withdrawal.

5. A method according to claim 2, wherein said disorder is associated with and/or attributed to allopregnanolone withdrawal.

6. A method according to claim 2, wherein said disorder is associated with and/or attributed to androstanediol withdrawal.

7. A method, according to claim 2, wherein said disorder is associated with and/or attributed to deoxycorticosterone, such as tetrahydrodeoxycorticosterone-(THDOC) withdrawal.

8. A method according to claim 1, said GABA_{A} receptor modulating steroid antagonist being 3beta-hydroxy-5alpha-pregnan-20-one, or a pharmaceutically acceptable salt thereof.

9. A method according to claim 1, said GABA_{A} receptor modulating steroid antagonist being 3beta-fluor-5alpha-pregnan-20-one, or a pharmaceutically acceptable salt thereof.

10. A method, according to claim 1, wherein the GABA_{A} receptor modulating steroid antagonist is administered in combination with one or more compounds selected from: acetaminophen, ibuprofen, acetylsalicylic acid, naproxen sodium, diclofenac potassium, almotriptan, eletriptan, frovatriptan, naratriptan, rizatriptan, sumatriptan, and zolmitriptan, dihydroergotamine, non-opioid acetaminophen, caffeine, metoclopramide, domperidone, prochlorperazine, etoprolol, propranolol, onabotulinumtoxin A, amitriptyline, venlafaxine, fluoxetine, topiramate, lamotrigine, gabapentin, carbamazepine, Erenumab and Fremanezumab-vfrm.

11. A method, according to claim 1, wherein said treatment is initiated no later than at a time point of onset of tolerance development to the substance or is initiated prior to the development of tolerance to the substance.

12. A method, according to claim 1, wherein said treatment is initiated at a time point prior to the expected manifestation of symptoms of said withdrawal disorder in a patient not undergoing treatment with GABAA receptor modulating steroid antagonist.

13. A method, according to claim 1, wherein said treatment is initiated at a time point at least about 1 day before the expected manifestation of symptoms of the substance withdrawal disorder.

14. A method, according to claim 1, wherein said treatment is initiated at a time point from about 2 to about 21 days prior to the expected manifestation of symptoms of the substance withdrawal disorder.

15. A method, according to claim 1, wherein said treatment is initiated at a time point of from about 10 to about 16 days prior to the expected manifestation of the symptoms of the substance withdrawal disorder.

16. A method, according to claim 1, wherein said treatment is initiated at a time point of from about 1 to about 6 months prior to the expected manifestation of the symptoms of the substance withdrawal disorder.

17. A method, according to claim 1, wherein said substance withdrawal disorder is headache, migraine, seizure(s), depression, anxiety and/or panic, associated with and/or attributed to allopregnanolone withdrawal.

18. A method according to claim 17, wherein said substance withdrawal disorder is headache and/or migraine associated with and/or attributed to allopregnanolone withdrawal.

19. A method according to claim 17, wherein said substance withdrawal disorder is a menstrual cycle related substance withdrawal disorder.

20. A method according to claim 19, wherein said expected manifestation of the substance withdrawal disorder coincides with onset of menstrual bleeding.

21. A method, according to claim 1, wherein said substance withdrawal disorder is headache, migraine, seizure(s), depression, anxiety and/or panic, associated with and/or attributed to THDOC withdrawal.

22. A method, according to claim 21, wherein said substance withdrawal disorder is a stress related substance withdrawal disorder.

23. A method according to claim 1, wherein said substance withdrawal disorder is headache, migraine, seizure(s), depression, anxiety and/or panic, associated with and/or attributed to androstanediol withdrawal.

24. A method, according to claim 23, wherein said substance withdrawal disorder is a season related substance withdrawal disorder.

25. A method according to claim 1, wherein the GABA_{A} receptor modulating steroid antagonist is administered in a pharmaceutical composition which further comprises one or more pharmaceutically acceptable carriers, excipients and/or diluents.

26. A method according to claim 25, wherein said GABA_{A} receptor modulating steroid antagonist is 3beta-hydroxy-5alpha-pregnan-20-one, or a pharmaceutically acceptable salt thereof.

27. A method according to claim 25, wherein said GABA_{A} receptor modulating steroid antagonist is 3beta-fluor-5alpha-pregnan-20-one, or a pharmaceutically acceptable salt thereof.
